(19) United States
(12) Patent Application Publication
(10) Pub. No.: US 2025/0213529 A1
(43) Pub. Date: Jul. 3, 2025

(54) 5-METHOXY-N.N-DIMETHYLTRYPTAMINE FOR THE TREATMENT OF PSYCHOMOTOR RETARDATION

(71) Applicant: GH RESEARCH IRELAND LIMITED, Dublin 2 (IE)

(72) Inventors: Theis TERWEY, Berlin (DE); Conor BURKE, Laytown (IE); Naoise GAFFNEY, Dublin (IE)

(73) Assignee: GH RESEARCH IRELAND LIMITED, Dublin 2 (IE)

(21) Appl. No.: 18/851,311

(22) PCT Filed: Mar. 27, 2023
(86) PCT No.: PCT/EP2023/057878

§ 371 Date: Sep. 26, 2024

(30) Foreign Application Priority Data
Mar. 27, 2022 (EP) 22000084.8
Mar. 27, 2022 (EP) 22000085.5
Jan. 30, 2023 (EP) 23153996.6
Jan. 30, 2023 (EP) 23154014.7

(51) Int. Cl. A61K 31/4045; A61K 9/00; A61P 25/18; A61P 25/24

(52) U.S. Cl. CPC A61K 31/4045; A61K 9/0019; A61P 25/18; A61P 25/24

(57) ABSTRACT

5-Methoxy-N,N-dimethyltryptamine (5-MeO-DMT) or a pharmaceutically acceptable salt thereof is used in treating a patient suffering from psychomotor retardation, wherein the 5-MeO-DMT or a pharmaceutically acceptable salt thereof is administered via the intra-venous, intramuscular or subcutaneous route.

### TECHNICAL FIELD

The present invention is directed to improved methods for the treatment of psychomotor retardation in a patient suffering from a mental disorder or a nervous system disorder, such as disorders characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome.

Psychomotor retardation can also occur in a patient suffering from sleep disturbance, for instance, insomnia.

The treatment comprises administering to a patient in need thereof a therapeutically effective amount of 5-methoxy-N,N-dimethyltryptamine (5-MeO-DMT) or of a pharmaceutically acceptable salt thereof.

### BACKGROUND OF THE INVENTION

Psychomotor retardation is characterized by reduced energy and activity and reduced motivation. Psychomotor retardation involves a slowing down of thought and a reduction of physical movements in an individual. Psychomotor impairment can cause a visible slowing of physical and emotional reactions.

Thus, there is a need for improved methods of treating psychomotor retardation, in particular psychomotor retardation associated with a mental disorder or a nervous system disorder.

### SUMMARY OF THE INVENTION

An aim of the invention is in particular the provision of improved therapies which are more effective (i.e., a) a larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies and dosing regimens for said therapies which have a better safety profile and/or are better tolerated than previously described therapies. Another aim of the current invention is to provide a compound for improved psychoactive therapies and dosing regimens for said therapies which are more convenient than previously described therapies. Another aim of the current invention is to provide a compound for improved psychoactive therapies and dosing regimens for said therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

The present invention provides 5-Methoxy-N,N-dimethyltryptamine (5-MeO-DMT) or a pharmaceutically acceptable salt thereof for use in treating a patient suffering from psychomotor retardation.

The present invention provides improved methods for the treatment of psychomotor retardation, in particular psychomotor retardation in a patient suffering from a mental or nervous system disorder, such as disorders characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome.

The invention also provides improved methods for the treatment of psychomotor retardation in a patient suffering from sleep disturbance, for instance, insomnia.

The present invention also provides dose ranges and dosing regimen useful for the treatment of psychomotor retardation.

In the context of the present invention, 5-MeO-DMT or a pharmaceutically acceptable salt thereof is administered via intravenous, intramuscular or subcutaneous administration.

5-MeO-DMT or a pharmaceutically acceptable salt thereof is administered at a dose or in a dosage regimen that causes the patient to experience a peak psychedelic experience. A dosage of about 1 mg to about 10 mg 5-MeO-DMT or an equimolar amount of a pharmaceutically acceptable salt may be administered.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a human subject who is suffering from psychomotor retardation by a licensed professional in accordance with accepted medical practice or who is diagnosed by a licensed professional in accordance with accepted medical practice with a mental disorder or a nervous system disorder associated with psychomotor retardation. In the latter case, assessing psychomotor retardation may or may not be part of the diagnosis.

Diagnosis of a mental disorder or a nervous system disorder can, for instance, be in accordance with the Diagnostic and Statistical Manual of Mental Disorders—Fifth Edition (DSM-5) published by the American Psychiatric Association. In some instances, as is apparent from the discussion of specific conditions below, the criteria may be modified or supplemented to better define patients or patient groups particularly benefiting from a treatment according to the invention. The diagnosis will in any event be by a physician or a psychologist. It is not sufficient that the human subject himself/herself considers that he/she is suffering from the disorder.

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

“Treatment of psychomotor retardation” shall include the management and care of a patient for the purpose of combating psychomotor retardation and includes the administration of compounds and methods according to the present invention to alleviate the signs and/or symptoms of psychomotor retardation or eliminate psychomotor retardation.

Psychomotor retardation may be associated with sleep disturbance, for instance, insomnia, a mental disorder or a nervous system disorder or another medical condition.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of the disease, for instance, if the patient suffers from a disorder characterized by depressive episodes, in the current episode of depression.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated.

“Clinical response” includes, but is not limited to, improvements on rating scales. These scales assess (i) psychomotor retardation or aspects of psychomotor retardation and/or (ii) a mental disorder or nervous system disorder or aspects of such a disorder and/or (iii) sleep.

The severity of a condition as well as changes of the severity can be assessed by the Clinical Global Impression (CGI) rating scales which are measures of symptom severity, treatment response and the efficacy of treatments.

The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

A clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

Alternatively, treatment success can be assessed using the CGI-Improvement (CGI-1), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

The Patient Global Impression scale (PGI), also known as Subject Global Impression (SGI), is the counterpart to the Clinical Global Impressions scale (CGI). It consists of one item based on the CGI and adapted to the patient. It can measure disease severity (PGIS) or disease improvement (PGI-I).

Individual items of scales as described herein as well as sub-combinations of individual items may be used to assess specific disease aspects.

As used in the context of the present invention, unless otherwise noted, the term “administration” (or “application”) shall mean the introduction of an amount, which may be a predetermined amount, of active compound or pharmaceutical ingredient into a patient via any route. The active compound is administered by intravenous administration, by intramuscular administration or by subcutaneous administration.

As used in the context of the present invention, unless otherwise noted, the terms “dose” and “dosage” and “dosage amount” shall mean the amount of active compound or pharmaceutical ingredient which is administered to a patient in an individual administration. The term “dosage regimen” (or “dosing regimen”) shall mean a defined sequence of one or more individual administrations.

### Psychomotor Retardation

Key aspects observed with patients suffering from psychomotor retardation are reduced energy and activity and reduced motivation.

Psychomotor retardation involves a slowing down of thought and a reduction of physical movements in an individual. Psychomotor impairment can cause a visible slowing of physical and emotional reactions.

Psychomotor retardation can be associated with a mental disorder or a nervous system disorder or some other medical conditions.

Mental or nervous system disorders which lead to, or are associated with, psychomotor retardation include disorders characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome.

Psychomotor retardation can also occur in a patient suffering from sleep disturbance, for instance, insomnia.

### Measuring Psychomotor Retardation

Psychomotor retardation can be assessed by measuring various aspects. These may include for instance various types of drawing tasks and tests, such as the trail making test (TMT), the digit symbol substitution test (DSST), or the Gibson Spiral Maze Test (GSM) and others which are known in the art.

In the trail making test (TMT), for instance, subjects must connect 25 circles that contain either numbers (TMT A) or a combination of numbers and letters (TMT B) in ascending order. Task requirements are similar for TMT-B, except that the subject must alternate between numbers and letters (1, A, 2, B, 3, C and so on). The test thus evaluates processing speed (TMT A) or cognitive flexibility (TMT B). The score for each part represents the amount of time required to complete the task.

Another test that involves graphomotor ability is the Gibson Spiral Maze (GSM) assessing psychomotor speed only, is not influenced by cognitive abilities. Subjects who complete the GSM must correctly trace through a spiral maze from a starting point to an end point without touching bordering lines.

The digit symbol substitution test (DSST) measures also psychomotor speed and consists of digit-symbol pairs followed by a list of digits. Under each digit, the subject should write down the corresponding symbol as fast as possible. The score consists in the number of symbols correctly reported in 90 s. A further example for a motor test is the finger tapping test.

Thus, certain tests combine measurements of both motor and cognitive aspects of psychomotor retardation, while further others assess only motor aspects.

Analysis of speech can be a further indicator of psychomotor retardation.

Major scales available to assess and measure include the severity of psychomotor retardation, the Salpêtrière Retardation Rating Scale (SRRS) and the Motor Agitation and Retardation Scale (MARS).

The Salpêtrière Retardation Rating Scale (SRRS), developed by Widlöcher assesses cognitive and motor aspects by fifteen items. The first three measure movement, specifically the quality of stride and slowness of limb, trunk, head, and neck movement. The next three items focus on speech including verbal flow, tone of voice, and length of response. Two items are designed to objectively measure cognitive function. These questions are based on the interview conversation and measure the patient's ability to approach and expand on topics. The further items are subjective and assess rumination, fatigue, level of interest, perception of time, memory, and concentration. The last item of the scale relates to an overall assessment of the patient's psychomotor retardation. The items are scaled from 0 (symptom absence) to 4 (severe) based on the severity of the presenting symptom, for a total score range of 0 to 60.

The Motor Agitation and Retardation Scale (MARS) assesses motor aspects only. It was designed to assess psychomotor disturbances in depressive disorders. Psychomotor disturbances are divided into five major body categories including eyes, face, voice, limbs, and trunk with a total of 19 items on the scale. Items of the eyes category include direction of gaze, amount of blinking, staring, and eye movement. Items associated with the face category include facial expression and facial expressivity. The category of voice has items that include volume, slurring, tone and time for onset. Items under the limbs category include hand, foot, and leg movement, stride, motor slowness, and tension in hands. The trunk category items include posture, immobility, and axial movement. The severity of each item ranges from a 1 to a 4, with 4 being the most severe. Of the 19 items 9 relate to motor agitation and 10 items assess motor retardation. The retardation items include abnormal gait, immobility of trunk/proximal limbs, postural collapse, motor slowness (i.e. the limb and trunk category); lack of facial expressivity, downcast gaze (i.e. the eyes and face category); and reduced voice volume, slurring of speech, delayed speech onset, monotone speech (i.e. the voice category). The MARS scale offers a rapid clinical assessment of motor signs.

### Scales to Assess Mental and Nervous System Disorders

Numerous scales have been suggested to assess severity of a mental disorder or a nervous system disorder. Such scales are based on tests which can be self-administered or administered by a clinician.

Scales which may be used according to the invention include those known in the art for diagnosis and/or monitoring the mental or nervous system disorders discussed in more detail below.

Treatment outcome is assessed by using one or more indices or scales at one or more time points after completion of a treatment course.

The assessment can be carried out after the acute psychedelic experience has subsided. An appropriate point in time for an early assessment is generally about 2 to 3 hours after the last administration. An early assessment can generally be carried out, for instance, about 2 hours or about 3 hours after the last administration.

An assessment of an effect on sleep disturbance can, however, be carried out at the earliest on the day after the treatment (i.e., on day 1) so that the treated patient had the opportunity to sleep for at least one night.

Thus, an assessment at day 1 or on day 1 means an assessment on the day following the administration. The assessment will be carried out not earlier than 12 hours after the last administration and in any event not earlier than one night after the last administration and not later than 36 hours after the last administration. The assessment can be carried out after about 24 hours.

An assessment at day 7 or on day 7 means an assessment on the seventh day following the administration (the day of administration is day 0). Analogous definitions apply for other assessment timings measured in days.

When assessing a clinical response, for instance, using one of the scales to assess severity of a mental disorder or a nervous system disorder, at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied. The same applies with respect to any other scale applied herein, unless a recall period is specifically indicated.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if necessary, an adapted recall period, so that it is not necessary to carry forward any pre-treatment score.

### Resting State Networks and Psychomotor Retardation

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system RSNs have been shown to be responsible for various aspects of complex brain function, and it has been found that these connectivity networks are compromised in various disease states. Such disease states, which include certain forms of psychomotor retardation, are associated with altered functional connectivity within a specific resting state network and/or between one or more regions in one or more additional resting state networks.

For instance, abnormal functional connectivity was reported from somatosensory motor networks (SMN) to visual (VN), dorsal attention (DAN), and default mode networks which correlated with both psychomotor retardation and agitation in depressive disorders.

In many instances, resting state networks involved in psychomotor retardation are affected by mental disorders or nervous system disorders, characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson Disease; Chronic Fatigue Syndrome.

Resting state networks involved in psychomotor retardation are also affected by sleep disturbance, for instance, insomnia. In fact, psychomotor retardation and impairment of sleep are correlated.

### Treatment of Psychomotor Retardation and Mental or Nervous System Disorders

According to the invention, psychomotor retardation in patients suffering from a mental disorder or a nervous system disorder can be treated. Moreover, psychomotor retardation occurring in a patient suffering from sleep disturbance, for instance, insomnia can be treated.

In patients suffering from psychomotor retardation in association with another condition as detailed above, a treatment of psychomotor retardation according to the invention leads to an improvement of the condition with which the psychomotor retardation is associated.

Treatment according to the invention is by administration of 5-MeO-DMT or a pharmaceutically acceptable salt thereof.

5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those resting state networks by a therapy as described herein will lead to an improvement of psychomotor retardation and, if the patient treated suffers from a mental disorder or a nervous system disorder, also of that disorder; if the patient treated suffers from sleep disturbance, for instance, insomnia, also of the sleep disturbance, for instance, insomnia.

To further support the clinical application of 5-MeO-DMT in patients suffering from psychomotor retardation, the inventors assessed clinical data relating to the use of 5-MeO-DMT in patients treated because of mental disease and noted particular improvements in psychomotor retardation which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with psychomotor retardation.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Åsberg Depression Rating Scale (MADRS). While the focus of the trial was on demonstrating treatment efficacy through improvements in overall MADRS score, the inventors focused on the items comprising the various rating scales and noticed that particular subscore items, like items related to psychomotor retardation, are relevant for other conditions in which psychomotor retardation is based on similarly altered functional connectivity within and/or between the somatomotor/sensorimotor network, the visual network, the dorsal attention network and the default mode networks.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is psychomotor retardation. 5-MeO-DMT can be administered to patients to reduce or eliminate psychomotor retardation in said patients.

The MADRS scale item that is of particular relevance to psychomotor retardation is “lassitude”, which represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort. A score of 6 is assigned in the case of complete lassitude, the patient being unable to do anything without help.

In the study group receiving the individualized dosing regimen, the aggregated score for the MADRS item “lassitude” across all 8 patients was 27 at base line.

After 2 hours, it was reduced to 10 which corresponds to an improvement of 17 points or 63%. At day 1 after treatment, it was reduced to 5 which corresponds to an improvement of 22 points or 81%. At day 7 after treatment, it was reduced to 3 which corresponds to an improvement of 24 points or 89%.

In the 12 mg group, the aggregated score for the MADRS item “lassitude” across all 4 patients was 16 at base line. After 2 hours, it was reduced to 10 which corresponds to an improvement of 6 points or 38%. At day 1 after treatment, it was reduced to 0 which corresponds to an improvement of 16 points or 100%. At day 7 after treatment, it was reduced to 3 which corresponds to an improvement of 13 points or 81%.

Thus, the score of the scale item that is of particular relevance to psychomotor retardation, “lassitude”, is markedly improved. The inventors conclude that 5-MeO-DMT can be used to treat psychomotor retardation in patients, in particular patients also suffering from a mental disorder or a nervous system disorder or a sleep disturbance, for instance insomnia.

Consequently, according to the invention, the treatment of a patient suffering from psychomotor retardation with 5-MeO-DMT or a pharmaceutically acceptable salt thereof reduces or eliminates psychomotor retardation.

### The Active Agent

The above discussion shows that psychomotor retardation as such present a significant disease burden and deserve appropriate treatment.

The inventors considered that a carefully chosen hallucinogen may lead to an improved treatment of important aspects of psychomotor retardation and may lead to overall improvements of the condition.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca, which contains DMT) in certain mental disorders suggest that those compounds could provide an alternative to the currently available treatments for certain mental disorders. However, there are reports that these compounds can induce mania in patients suffering from depressive symptoms, and this may preclude their clinical use.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138(11):1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23(4):1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyltryptamine (DMT). Journal of Psychoactive Drugs, 49(4), 294-296.

The inventors considered that in order to avoid the induction of mania or hypomania or at least reduce the risk of induction of mania or hypomania, the compound administered must be appropriately chosen and preferably is administered in a particular dosing regimen.

The inventors identified 5-methoxy-N,N-dimethyltryptamine (5-MeO-DMT) as a psychedelic of particular interest for use in therapy. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia; or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.”-Journal of Psychiatric Practice-13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after intravenous injection compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

Furthermore, 5-MeO-DMT is a 5-HT7 receptor agonist showing high affinity towards the receptor. The inventors determined, using recombinant human 5-HT7 receptor, [³H]LSD as a radio ligand and serotonin to estimate non-specific binding, a Kᵢ of 2.3 nM.

Thus, besides the 5-HT1A and 5-HT2A receptors discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, negative thinking, sleep disturbance or social/emotional withdrawal. This is supported by the clinical results demonstrated in studies referred to herein.

Another feature of 5-MeO-DMT is its short half-life.

5-MeO-DMT is mainly inactivated through a deamination pathway mediated by monoamine oxidase A, and it is O-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

The properties of 5-MeO-DMT make the compound especially suitable for the treatment of psychomotor retardation, in particular for patients suffering from a mental disorder or a nervous system disorder.

The properties of 5-MeO-DMT also allow specific dosage regimens, as discussed in more detail below.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, this can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoroacetate) and 5-MeO-DMT methyl pivalate (3-(2-(dimethylamino)ethyl)-5-methoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

### Modes of Administration

The therapeutically effective amount of 5-MeO-DMT is administered by intravenous administration, by intramuscular administration or by subcutaneous administration. Administration via these routes can assure a rapid onset of action. A most preferred route of administration is administration via the intravenous route, i.e. by intravenous injection. 5-MeO-DMT can be employed as a pharmaceutically acceptable salt, preferably the hydrobromide salt, or in the form of a formulation for administration via injection, examples of excipients and vehicles for such formulations being known in the art.

### Dosing Regimen

The present invention also provides dose ranges, particular doses as well as dosing regimens (administration schemes) and appropriate routes of administration.

The invention is in part based on the inventors' conclusion that the occurrence of a peak psychedelic experience during the acute phase after administration of 5-MeO-DMT or a pharmaceutically acceptable salt thereof is driving its therapeutic benefit in patients suffering from psychomotor retardation, in particular one or more of the aspects defined above, either in a causal relationship or at least as a surrogate behavioral marker for the underlying unknown therapeutic mechanism.

Consequently, achieving peak experiences more rapidly, in a larger proportion of patients and with better reproducibility in an individual patient, compared with previously tested psychedelic agents, dosing regimens and administration routes, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flashbacks of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein who suffers from psychomotor retardation is treated by administration of 5-MeO-DMT or a pharmaceutically acceptable salt thereof.

In a preferred embodiment, the 5-MeO-DMT is administered as a monotherapy, i.e., the patient does not receive any other treatment for psychomotor retardation.

The dosage amount of 5-MeO-DMT administered to a patient, as defined herein, suffering from psychomotor retardation, is in the range of about 1 mg to about 10 mg, or any amount of range therein and is administered in the form of a formulation for administration based on a pharmaceutically acceptable salt of 5-MeO-DMT, such as the hydrobromide salt, which weight amount can be calculated from the stated weight amounts of the 5-MeO-DMT free base, assuming that equimolar amounts are used. Useful specific amounts of 5-MeO-DMT are e.g. about 1 mg, about 2 mg, about 3 mg, about 4 mg, about 5 mg, about 6 mg, about 7 mg, about 8 mg, about 9 mg, and about 10 mg. Note that in this specification, when ranges are set forth, such as “about 1 mg to about 10 mg,” the inventor contemplates all discrete values within that range, some of which are specifically mentioned, but all of which are not—simply for the purpose of brevity.

In preferred embodiments the improved methods for the treatment of a patient, as defined herein, suffering from psychomotor retardation, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, suffering from psychomotor retardation, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, suffering from psychomotor retardation, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 10 mg.

In a preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered.

In an even more preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 0.25 mg to about 3 mg, preferably about 0.5 mg to about 3 mg to the dosage amount of the prior administration. For example, if the dosage amount of the first administration was 1 mg and the dosage amount increase is 3 mg, unless one of the previously mentioned stopping criteria has been reached, then the dosage amount of the second administration will be 4 mg. Preferably, the dosage amount for the third administration will be 7 mg.

In a preferred embodiment the dosage amount of the 5-MeO-DMT administered to an individual patient in each treatment block is selected from about 1 mg to about 3 mg for the first administration, and then increased, unless the patient has already experienced a peak psychedelic experience within that treatment block or the supervising physician has decided that further dose increases are inappropriate based on observed side effects, to a dosage selected from about 4 mg to about 6 mg for the second administration, and from about 7 mg to about 9 mg for the third administration. Useful specific amounts for the first, second and third administration are e.g. about 2 mg, about 5 mg, and about 8 mg.

In an additional preferred embodiment, the dosage amount of the 5-MeO-DMT administered to an individual patient in each treatment block is selected from about 0.5 mg to about 1.5 mg for the first administration, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration, and from about 2.5 mg to about 3.5 mg for the third administration. Useful specific amounts for the first, second and third administration are e.g. about 1 mg, about 2 mg, and about 3 mg.

In a further preferred embodiment the dosage amount of the 5-MeO-DMT administered to an individual patient is selected from about 1 mg to about 2 mg for the first administration of the first treatment block, and then increases with each subsequent administration within that first treatment block until the earlier of 10 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 8 mg because the patient experienced a peak psychedelic experience at that dose, then the dosage for all subsequent treatment blocks and administrations within those subsequent treatment blocks will be 8 mg.

In a particularly preferred embodiment the dosage amount of the 5-MeO-DMT administered to an individual patient is selected from about 1 mg to about 3 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 7 mg to about 9 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2 mg, about 5 mg, and about 8 mg.

In a particularly preferred embodiment the dosage amount of the 5-MeO-DMT administered to an individual patient is selected from about 0.5 mg to about 1.5 mg for the first administration of the first treatment block, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration of the first treatment block, and from about 2.5 mg to about 3.5 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 1 mg, about 2 mg, and about 3 mg.

It is understood that a pharmaceutically acceptable salt of 5-MeO-DMT is preferably used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett F S, J Psychopharmacol. 2015; 29(11):1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8:974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?

### Treatment of Psychomotor Retardation and Sleep Disturbance

Sleep disturbance refers to conditions, whether idiopathic or occurring in the context of a medical condition such as for example a mental disorder or a nervous system disorder, that affect sleep quality, timing, or duration. It impacts a person's ability to properly function while the person is awake and is linked to a pattern of high negative emotions and low positive emotions.

There are two fundamental types of sleep: rapid eye movement (REM) sleep and non-REM sleep. Non-REM sleep can be divided into four stages (I-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night.

Disruption of this tight regulation results in sleep disturbances.

Common forms of sleep disturbances encompass disorders of initiating and maintaining sleep (insomnia), disorders of excessive somnolence (hypersomnia), disorders of slee-pwake schedule (circadian rhythm disorders), dysfunctions associated with sleep, sleep stages, or partial arousals (parasomnia), disorders characterized by respiratory disturbance during sleep (sleep-related breathing disorders) and disorders characterized by abnormal movements during sleep (sleep-related movement disorders). In a broad sense, fatigue can also be considered a sleep disturbance.

Insomnia is a sleep disturbance where people have difficulty falling or staying asleep.

People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterized by excessive daytime sleepiness, and/or prolonged nighttime sleep. Sleep drunkenness is also a symptom found in hypersomnia patients. It is a difficulty transitioning from sleep to wake. Individuals experiencing sleep drunkenness report waking with confusion, disorientation, slowness and repeated returns to sleep.

Circadian rhythm disorders are characterized by chronic or recurring sleep disturbances due to alterations of the individual's internal circadian rhythm or due to misalignments between their circadian rhythm and their desired or required work or social schedule. This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterized by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterized by abnormal and difficult respiration during sleep. Respiration is a complex process that relies heavily on the coordinated action of the muscles of respiration and the brain. With a potentially serious impact on sleep and the balance of oxygen and carbon dioxide in the blood, these sleep disturbances appear as chronic snoring, sleep apnoea, sleep related hypoventilation and/or hypoxemia. In some of these disorders, respiration is also abnormal during wakefulness. The reduction of airflow leads to intermittent hypoxia, with microarousals or awakenings, causing sleep fragmentation and excessive daytime sleepiness. Inflammation and endothelial dysfunction may ensue and reduce vascular elasticity and increase coagulation predisposing individuals to atherosclerosis, which, along with reduced oxygenation, may cause heart and brain damage.

In sleep-related movement disorders repetitive, relatively simple, usually stereotyped, movements interfere with sleep or its onset. The most common of these are restless leg syndrome (RLS) and periodic limb movement disorder (PLMD).

Fatigue describes a state of tiredness that does not resolve with rest or sleep. It is a feeling of exhaustion, lethargy, or decreased energy, usually experienced as a weakening or depletion of one's physical or mental resource and characterised by a decreased capacity for work and reduced efficiency in responding to stimuli. Fatigue is normal following a period of exertion, mental or physical, but sometimes may occur in the absence of such exertion as a symptom of health conditions.

Not getting the proper amount or quality of sleep may lead to personality changes and may not only exacerbate existing mental illness, but also be a trigger for the development of mental illness.

Elderly people are at particular risk for sleep disorders such as insomnia and circadian rhythm abnormalities. Severe and persistent sleep disturbances in older patients may result in neuropsychiatric symptoms such as psychomotor retardation. Sleep loss can also adversely affect life by contributing to the development of obesity, diabetes and heart disease.

Treatment of sleep disturbance varies depending on the type and underlying cause. Maintenance of good sleep hygiene, a healthy sleep environment, and a consistent sleep-wake schedule are often considered as first-line treatment. If not successful, treatment also involves pharmacotherapy or psychotherapy.

Available treatments are not successful in all patients, may be associated with side effects and/or require treatment over a long period of time to achieve a relevant treatment effect.

In patients suffering from sleep disturbance in association with a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment (Hutka et al. Association of Sleep Architecture and Physiology with Depressive Disorder and Antidepressants Treatment. Int J Mol Sci. 2021 Jan. 29; 22(3):1333., Abstract).

To assess sleep, parameters such as sleep duration, sleep architecture, sleep latency, and the frequency and duration of awakenings throughout the night can be measured. The quantitative metrics may be measured using objective methods, including polysomnography, actigraphy, and the determination of sleep latency, or by way of self-reported measures (questionnaires).

Polysomnography is a technique requiring that a patient is monitored overnight at a specialized clinic. A variety of functions are measured throughout the night, including eye movements, brain and muscle activity, respiratory effort and airflow, blood oxygen levels, body positioning and movements, snoring, and heart rate.

Sleep latency can be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance. Accompanying analysis of brain activity can assist in the further diagnose of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use.

The evaluation of the qualitative aspects of sleep experience is important, as sleep complaints can often persist despite normal values for quantitative measures of sleep.

Questionnaires not only facilitate a quick and accurate assessment of a complex clinical problem, but they are potentially also helpful for tracking a patient's progress. Thus, self-reported sleep questionnaires completed by patients are an important mainstay for the assessment of sleep disturbance in clinical practice.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Sleep quality in general can be assessed, for instance, with Sleep Quality Scale (SQS) and the Sleep-50 questionnaire.

The Sleep Quality Scale (SQS) is an all-inclusive assessment tool to achieve a general, efficient measure suitable for evaluating sleep quality in a variety of patient and research populations. Individual questions on daytime symptoms, such as attention, concentration difficulties or memory problems (Item 15, “Poor sleep makes hard for me to think”, item 19 “Poor sleep causes me to make mistakes at work”, item 21 “Poor sleep makes me forget things more easily”, item 22 “Poor sleep makes it hard to concentrate at work”), restoration after sleep, problems initiating and maintaining sleep, difficulty waking, and sleep satisfaction can be scored from 0 (“rarely”)-3 (“almost always”), with higher scores being indicative for more acute sleep problems.

The SLEEP-50 questionnaire consists of 50 items designed to screen for various kinds of sleep disturbance in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

The questionnaire requires that for diagnosing a sleep disturbance not only the specific subscale (e.g., insomnia) exceeds a certain cut-off point, but also the impact sub-scale (Spoormaker et al. Initial validation of the SLEEP-50 questionnaire. Behav Sleep Med. 2005; 3(4):227-46., table 4 for optimal cut-off values and scoring procedures).

Treatment success is indicated (i) by a decrease of the score, preferably (ii) by a decrease to below the cut-off value.

A common questionnaire assessing sleep disturbance is the Pittsburgh Sleep Quality Index. Other instruments are the insomnia severity index and the Espie sleep disturbance questionnaire.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window.

The 19 self-rated questions assess a wide variety of factors relating to sleep quality, including estimates of sleep duration and latency and of the frequency and severity of specific sleep-related problems. These 19 items are grouped into seven component scores: (1) subjective sleep quality; (2) sleep latency; (3) sleep duration; (4) habitual sleep efficiency; (5) sleep disturbances; (6) use of sleeping medication; (7) daytime dysfunction.

Each component is assigned a score of 0 to 3. Higher scores indicate more acute sleep disturbances. Detailed Scoring Instructions for the Pittsburgh Sleep Quality Index can be found in the Appendix of Buysse et al. The Pittsburgh Sleep Quality Index: a new instrument for psychiatric practice and research. Psychiatry Res. 1989 May; 28(2):193-213.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient is having severe difficulties in at least two areas, or moderate difficulties in more than three areas.

If treatment outcome is assessed using the PSQI, treatment success is indicated (i) by a decrease of the score, preferably (ii) by a decrease to 5 or below.

The Insomnia Severity Index (ISI) is a 5-item questionnaire relating to subjective sleep quality, severity of symptoms, subjective satisfaction with sleep, the degree to which insomnia interferes with daily functioning (Item 3, “To what extent do you consider your sleep problem to interfere with your daily functioning (e.g. daytime fatigue, ability to function at work/daily chores, concentration, memory, mood, etc).”), how noticeable the respondent feels his or her insomnia is compared to others, and the overall level of distress created by the sleep problem. Individual responses can be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)” (A. Shahid et al. (eds.), STOP, THAT and One Hundred Other Sleep Scales, Springer Science+Business Media, LLC 2012; Bastien et al. Validation of the Insomnia Severity Index as an outcome measure for insomnia research. Sleep Med. 2001 July; 2(4):297-307).

The usual recall window for the ISI is two weeks, but other appropriate recall windows can also be used herein.

Treatment success is indicated (i) by a decrease of the score, for instance, by >7 points, in particular >8 point; preferably (ii) by a decrease to below the cut-off value for clinically significant insomnia.

The Espie sleep disturbance questionnaire (SDQ) evaluates subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia (A. Shahid et al., loc. cit.; Espie et al. Insomniacs' attributions. psychometric properties of the Dysfunctional Beliefs and Attitudes about Sleep Scale and the Sleep Disturbance Questionnaire. J Psychosom Res. 2000 February; 48(2):141-8).

Treatment success is indicated by a decrease of the score.

Hypersomnia or hypersomnolence can be assessed by the Epworth sleepiness scale, the Stanford sleepiness scale, or the idiopathic hypersomnia severity scale.

The Epworth sleepiness scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level (A. Shahid et al., loc. cit. Johns et al., A new method for measuring daytime sleepiness: the Epworth sleepiness scale. Sleep, 1991 December; 14(6):540-5).

Treatment success is indicated (i) by a decrease of the score, preferably (ii) by a decrease to 10 or below.

The Stanford sleepiness scale is a subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness (A. Shahid et al., loc. cit.; Hoddes et al. The development and use of the Stanford sleepiness scale (SSS). Psychophysiology, 1972, 9, 150).

Treatment success is indicated by a decrease of the score.

Parasomnias can be evaluated by the Paris Arousal Disorders Severity Scale (PADSS).

The Paris Arousal Disorders Severity Scale (PADSS) is a self-rating scale listing parasomniac behaviours, assessing their frequency and includes an evaluation of consequences (Arnulf et al. A scale for assessing the severity of arousal disorders. Sleep. 2014 Jan. 1; 37(1):127-36).

Treatment success is indicated (i) by a decrease of the score, preferably (ii) by a decrease to below the cut-off value.

A common questionnaire that assesses sleep-related breathing disorders is the Berlin Questionnaire (A. Shahid et al., loc. cit.; Netzer et al., Using the Berlin Questionnaire to identify patients at risk for the sleep apnea syndrome. Ann Intern Med. 1999 Oct. 5; 131(7):485-91). An appropriate recall period can also be chosen.

Treatment success is indicated by a decrease of the score.

A common questionnaire that assesses sleep-related movement disorders is the International Restless Legs Syndrome Study Group Rating Scale (A. Shahid et al., loc. cit.; Walters et al.; International Restless Legs Syndrome Study Group 2003. Validation of the International Restless Legs Syndrome Study Group rating scale for restless legs syndrome. Sleep Medicine 4(2), pp. 121-132).

Treatment response can be assessed by a decrease of the score.

Fatigue is commonly assessed by for example the FACES (Fatigue, Anergy, Consciousness, Energy, Sleepiness).

The FACES (Fatigue, Anergy, Consciousness, Energy, Sleepiness) rating scale is a 50 items checklist to distinguish between tiredness, sleepiness, and fatigue. Respondents use a scale ranging from 0 (“not at all”) to 3 (“strongly”) to indicate the degree to which they have experienced each feeling or energy state over the course of the previous week or another appropriate recall period. Higher scores indicate more acute states of tiredness or fatigue, except for those items belonging to the energy subscale (A. Shahid et al., loc. cit.; Shapiro et al., Development of an adjective checklist to measure five FACES of fatigue and sleepiness. Data from a national survey of insomniacs. J Psychopomp Res. 2002 June; 52(6):467-73).

Effective treatment reduces scoring in tiredness and/or fatigue and/or increases scoring on the energy sub-scale.

Treatment response can be evaluated by above-mentioned quantitative measurements such as polysomnography or actigraphy and/or the use of the above-mentioned questionnaires. Depending on the respective sleep scale, a significant reduction or increment, respectively, in total score, or significant reduction of prevalence, frequency and impact on daily-functioning, respectively is indicative for treatment-induced improvement of the sleep disturbance.

A common test that assesses psychomotor retardation or aspects thereof that can be used is the Salpêtrière Retardation Rating Scale (SRRS).

To enhance the understanding of the pathophysiology and potential compensatory mechanisms at play, resting-state fMRI has been widely applied in patients with sleep disturbances.

Altered resting state networks can be found in insomnia, hypersomnia, circadian rhythm disorders, parasomnias, sleep-related breathing disorders and sleep-related movement disorders.

In insomnia patients, dysfunctional connectivity is observed within the default mode network (DMN) and within the salience network, which is implicated in the detection and integration of emotional and sensory stimuli. Studies have suggested that these networks contain critical regions integrating emotional and bodily states, and the dysfunctional connectivity within and/or between these networks and other brain areas may underlie the vigilance, subjective distress, and poor sleep continuity of patients.

For instance, sleep deprivation in healthy subjects leads to functional connectivity alterations within and/or between the default mode network, dorsal attention network and salience network and these brain functional connectivity changes somewhat resemble the vulnerability patterns of patients with Alzheimer's disease.

In hypersomnia patients, the default mode network is affected. For instance, in idiopathic hypersomnia, distinct DMN hubs—the precuneus and medial prefrontal cortex—demonstrate significant changes, and functional connectivity in the DMN correlates with self-reported sleepiness severity.

A study investigating differences between night shift nurses and day work nurses revealed that dysrhythmia of circadian rhythms contributes to resting-state functional changes in the cerebellum, involved in sleep regulation, and cognitive functions such as responsiveness and alertness. Moreover, the functional connectivity of the DMN is fundamentally different in early and late circadian phenotypes. Like other forms of sleep disturbance, circadian rhythm disorders can lead to changes in brain functional connectivity. Changes in resting state brain functional connectivity have been reported in various diseases with circadian rhythm disorders.

While functional brain imaging is technically difficult to perform during a parasomnia event, differences in the precuneus have been observed in disorders of arousal representing a non-REM parasomnia.

The precuneus is involved in the analysis and integration of visual, audio, and somesthetic information and the monitoring of movements. The precuneus is a subregion of the DMN. Thus, in patients with parasomnias the default mode network is affected.

Resting-state fMRI studies in patients suffering from sleep-related breathing disorders, such as obstructive sleep apnoea (OSA), have demonstrated that long-term exposure to oxidative stress, intermittent hypoxia, hypo- and hypercapnia and sleep fragmentation, some of the major causes of OSA brain injury, may lead to significant global and regional connectivity deficits, especially in the default mode network (DMN) and regions involved in the arousal and sensorimotor systems.

Sleep-related movement disorders, such as for example periodic limb movements during sleep are reflected by alterations in the prefrontal motor control pathway, a subregion of the default mode network. Also, activity in the cerebellum and thalamus, with additional activation in the red nuclei and brainstem can be observed.

In many instances, aberrant functional connectivity of resting state networks involved in sleep regulation are also involved in psychomotor retardation. Thus, according to the invention, influencing those networks by a therapy according to the invention will lead to an improvement of the sleep disturbance and, if the patient treated suffers from psychomotor retardation, also of the psychomotor retardation.

Clinical data from studies of patients suffering from TRD or Postpartum Depression (PPD) confirm that sleep disturbance can successfully be treated.

In the studies, which involved the administration of 5-MeO-DMT, among others the MADRS item “reduced sleep”, which reflects insomnia, was assessed.

The MADRS item “reduced sleep” represents the experience of reduced duration or depth of sleep compared to the subject's own normal pattern when well. A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

Thus, the score of the scale item that is of particular relevance to sleep disturbance, “reduced sleep”, is markedly improved. The inventors conclude that 5-MeO-DMT can be used to treat sleep disturbance, in particular patients suffering from a mental disorder or a nervous system disorder.

Treating a patient suffering from sleep disturbance, in particular insomnia, and associated psychomotor retardation with 5-MeO-DMT or a pharmaceutically acceptable salt thereof reduces or eliminates the psychomotor retardation and leads to an improvement of the sleep disturbance, in particular the insomnia.

The reduction or elimination of psychomotor retardation in a patient suffering from sleep disturbance, in particular insomnia, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from sleep disturbance, in particular insomnia, occurs not later than about 2 hours after the last administration of 5-MeO-DMT or a pharmaceutically acceptable salt thereof. The reduction or elimination of psychomotor retardation in a patient suffering from sleep disturbance, in particular insomnia, preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from sleep disturbance, in particular insomnia, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from sleep disturbance, in particular insomnia, as reflected by at least an improvement in the score of the Salpêtrière Retardation Rating Scale (SRRS) occurs not later than about 2 hours after the last administration of 5-MeO-DMT or a pharmaceutically acceptable salt thereof. The reduction or elimination of psychomotor retardation in a patient suffering from sleep disturbance, in particular insomnia, as reflected by at least an improvement in the score of the Salpêtrière Retardation Rating Scale (SRRS) preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from a sleep disturbance. An improvement in psychomotor retardation will therefore also lead to an improvement of the sleep disturbance. Since psychomotor retardation furthermore also affects other aspects of the sleep disturbance, the inventors conclude that the improvement in psychomotor retardation will additionally contribute to an overall improvement of the sleep disturbance, in particular insomnia.

In cases of idiopathic sleep disturbance in a patient also suffering from associated psychomotor retardation, a clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

An improvement of idiopathic sleep disturbance in a patient also suffering from associated psychomotor retardation, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of idiopathic sleep disturbance in a patient also suffering from associated psychomotor retardation, as reflected by a reduction in the CGI-S score in a patient also suffering from associated psychomotor retardation, occurs not later than about 24 hours after the last administration of 5-MeO-DMT or a pharmaceutically acceptable salt thereof. An improvement in idiopathic sleep disturbance in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance in a patient also suffering from associated psychomotor retardation, as reflected by a reduction in the CGI-S score, is observed on day 7 after the last administration of 5-MeO-DMT or a pharmaceutically acceptable salt thereof and preferably persists until at least 14 days, more preferably until at least 28 days.

In cases of idiopathic sleep disturbance in a patient also suffering from associated psychomotor retardation, an improvement in sleep disturbance, as reflected by at least a score of “much improved” in the Clinical Global Impression—Improvement (CGI-1) score or the Patient Global Impression—Improvement (PGI-I) score, preferably occurs not later than about 24 hours after the last administration of 5-MeO-DMT or a pharmaceutically acceptable salt thereof.

In cases of idiopathic sleep disturbance in a patient also suffering from associated psychomotor retardation, an improvement in sleep disturbance, as reflected by a reduction by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

Improvements in cases of idiopathic sleep disturbance in a patient also suffering from associated psychomotor retardation may also be assessed by any other scale reflecting changes in sleep quality or quantity, as indicated above, for instance the Pittsburgh Sleep Quality Index (PSQI).

If treatment outcome is assessed using the PSQI, treatment success is indicated (i) by a decrease of the score, preferably (ii) by a decrease to 5 or below.

An improvement in idiopathic sleep disturbance in a patient also suffering from associated psychomotor retardation, as reflected by a decrease of the PSQI score, in particular by a decrease to 5 or below, preferably occurs not later than about 24 hours after the last administration of 5-MeO-DMT or a pharmaceutically acceptable salt thereof, wherein the recall period spans from the time point when acute psychedelic experiences have subsided after the last administration to the assessment time point.

Such an improvement in idiopathic sleep disturbance in a patient also suffering from associated psychomotor retardation, as reflected by a decrease of the PSQI score, preferably persists until at least 6 days; in particular until at least 14 days; more preferably until at least 28 days after the last administration of 5-MeO-DMT or a pharmaceutically acceptable salt thereof, wherein the recall period spans from the time point when acute psychedelic experiences have subsided after the last administration to the assessment time point.

An improvement of idiopathic sleep disturbance in a patient also suffering from associated psychomotor retardation, as reflected by a reduction in the PSQI score, is observed on day 1, for instance after about 24 hours; on day 7; on day 14; and/or on day 28 after the last administration of 5-MeO-DMT or a pharmaceutically acceptable salt thereof, wherein the recall period spans from the time point when acute psychedelic experiences have subsided after the last administration to the assessment time point.

Psychomotor retardation and sleep disturbance, in particular insomnia, are closely linked to mental and nervous system disorders. Both, psychomotor retardation and sleep disturbance, in particular insomnia, occur in mental or nervous system disorders, such as a disorder characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease (PD); Chronic Fatigue Syndrome.

A treatment according to the invention will lead to improvements in both psychomotor retardation and sleep disturbance, in particular insomnia, and furthermore to an improvement in the associated mental or nervous system disorder, examples of which are listed above.

### Treatment of Psychomotor Retardation and Mental and Nervous System Disorders

In patients suffering from psychomotor retardation in association with a mental disorder or a nervous system disorder a treatment of psychomotor retardation according to the invention leads to an improvement of the condition with which the psychomotor retardation is associated.

While psychomotor retardation may be considered a condition deserving treatment independent of any other conditions, disorders or symptoms an individual may suffer from, several mental disorders and disorders of the nervous system are associated with psychomotor retardation. Notably, the relationship between psychomotor retardation and mental disorder is bidirectional. Not only can mental disorders have a negative impact on psychomotor retardation in the patient, but psychomotor retardation can also be a contributing factor to the onset, progression and prognosis of mental or nervous system disorders.

In many instances, resting state networks involved in psychomotor retardation are also involved in the conditions listed above.

5-MeO-DMT has the ability to disrupt established functional connectivity patterns of resting state networks. This disruption leads to a reset of the pathological ill-connected brain connections as the networks reconnect. New, healthy functional connections are established with persistent effects.

### Disorders Characterized by Depressive Episodes

There are several disorders which are characterized by depressive episodes.

A depressive episode is a period of depressed mood and/or loss of pleasure in most activities.

For instance, according to DSM-V, a Major Depressive Episode is characterized by five or more symptoms that have been present during the same 2-week period and represent a change from previous functioning; at least one of the symptoms is either (1) depressed mood or (2) loss of interest or pleasure, and including symptoms such as psychomotor retardation.

The patient suffering from a disorder characterized by depressive episodes may suffer from a treatment resistant form of the disorder.

Psychomotor retardation occurs in patients suffering from disorders characterized by depressive episodes.

Psychomotor retardation is included as a diagnostic criterion for disorders characterized by depressive episodes. It may be assessed as part of the determination of the HAM-D or the MADRS.

The Montgomery-Åsberg Depression Rating Scale (MADRS) is a ten-item diagnostic questionnaire used to measure the severity of depressive episodes in patients with mood disorders. Higher MADRS scores indicate more severe depression.

Items are apparent sadness; reported sadness; inner tension; reduced sleep; reduced appetite; concentration difficulties; lassitude; inability to feel; pessimistic thoughts; and suicidal thoughts and each item yields a score of 0 to 6. The overall score ranges from 0 to 60.

The MADRS item “lassitude” represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort. A score of 6 is assigned in the case of complete lassitude, the patient being unable to do anything without help.

This MADRS scale item is of particular relevance to psychomotor retardation, i.e., reduced energy and activity and reduced motivation.

The Hamilton Rating Scale for Depression (HAM-D) allows clinicians to assess the nature and severity of mood disorders in patient populations. The scale is comprised of 21 items for inquiry, though only the first 17 (depressed mood; feelings of guilt; suicide; initial insomnia; insomnia during the night; delayed insomnia; work and interests; retardation; agitation; psychiatric anxiety; somatic anxiety; gastrointestinal somatic symptoms; general somatic symptoms; genital symptoms; hypochondriasis; weight loss; insight) are used in scoring.

For the majority of questions, scores range from 0 to 4, with 4 representing more acute signs of depression. Several questions have ranges that extend only as high as 2. A total score is tallied and can then be compared with previous scores or can be contrasted with a pre-defined cut-off score.

In the context of the item “retardation”, the questionnaire assesses slowness of thought and speech, impaired ability to concentrate, decreased motor activity.

Psychomotor retardation in a patient suffering from a disorder characterized by depressive episodes can, for instance, be further assessed using the Salpêtrière Retardation Rating Scale (SRRS).

In patients suffering from a disorder characterized by depressive episodes altered functional connectivity was observed within and/or between several brain regions implicated in processing, regulation, emotional memory; cognitive processes related to rumination; impaired concentration and physiological arousal.

Treating a patient suffering from a disorder characterized by depressive episodes, including a treatment resistant form of the disorder, and associated psychomotor retardation with 5-MeO-DMT or a pharmaceutically acceptable salt thereof reduces or eliminates the psychomotor retardation and leads to an improvement of the disorder characterized by depressive episodes.

The reduction or elimination of psychomotor retardation in a patient suffering from a disorder characterized by depressive episodes is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from a disorder characterized by depressive episodes occurs not later than about 2 hours after the last administration of 5-MeO-DMT or a pharmaceutically acceptable salt thereof. The reduction or elimination of psychomotor retardation in a patient suffering from a disorder characterized by depressive episodes, preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from a disorder characterized by depressive episodes, in particular of lassitude, is reflected by an improvement at least in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from a disorder characterized by depressive episodes, in particular of lassitude, as reflected by an improvement in the score of the MADRS item “lassitude”, occurs not later than about 2 hours after the last administration of 5-MeO-DMT or a pharmaceutically acceptable salt thereof. The reduction or elimination of psychomotor retardation in a patient suffering from a disorder characterized by depressive episodes, as reflected by an improvement in the score of the MADRS item “lassitude” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from a disorder characterized by depressive episodes, in particular of retardation, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from a disorder characterized by depressive episodes, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from a disorder characterized by depressive episodes, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from a disorder characterized by depressive episodes, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering a disorder characterized by depressive episodes, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering a disorder characterized by depressive episodes, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from a disorder characterized by depressive episodes, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering a disorder characterized by depressive episodes, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering a disorder characterized by depressive episodes, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to disorder characterized by depressive episodes. An improvement in psychomotor retardation will therefore also lead to an improvement of the disorder characterized by depressive episodes. Since psychomotor retardation furthermore also affects other aspects of a disorder characterized by depressive episodes, the inventors conclude that the improvement in psychomotor retardation, will additionally contribute to an overall improvement of the disorder characterized by depressive episodes.

An improvement of the disorder characterized by depressive episodes in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the disorder characterized by depressive episodes in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the disorder characterized by depressive episodes in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Major Depressive Disorder (MDD) is a mood disorder that causes a persistent feeling of sadness and loss of interest. It affects how a person feels, thinks and behaves and can lead to a variety of emotional and physical problems.

The patient suffering from MDD may suffer from a treatment resistant form of the disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of depression.

Psychomotor retardation is a core feature of Major Depressive Disorder (MDD), and is one of the 9 symptoms listed to identify MDD in the DSM-V.

Clinical manifestations of psychomotor retardation in MDD include speech abnormalities, reduction in eye contact and eye movements, and poor posture amongst others.

The presence of psychomotor retardation may be useful in guiding therapy planning, as there may be evidence that tricyclic antidepressants (TCAs) could provide greater response than selective serotonin reuptake inhibitors (SSRIs) in those cases.

Psychomotor retardation may be associated with greater symptom severity. A study of 291 patients with MDD found that those with greater life-time psychomotor retardation scores had more severe depression.

Psychomotor retardation in a patient suffering from MDD may be assessed as part of the determination of the HAM-D, or the MADRS. Psychomotor retardation or at least aspects thereof may furthermore be assessed, for instance, by using the Salpêtrière Retardation Rating Scale (SRRS) or the Motor Agitation and Retardation Scale (MARS).

MDD can be characterized as a disorder with dysfunctional connectivity and regulation within and/or between multiple resting-state networks including the DMN, salience network, executive control network, limbic network and the somatomotor network. Functional connectivity differs significantly from that observed in healthy controls.

Treating a patient suffering from MDD, including a treatment resistant form of the disorder, and associated psychomotor retardation with 5-MeO-DMT or a pharmaceutically acceptable salt thereof reduces or eliminates psychomotor retardation and leads to an improvement of MDD.

The patient may suffer from moderate or severe MDD as indicated by a Montgomery-Åsberg Depression Rating Scale (MADRS) score of 20 or more or by a Hamilton Depression Rating Scale (HAM-D) score of 17 or more. It is further considered that the patient may suffers from severe major depressive disorder as indicated by a Montgomery-Åsberg Depression Rating Scale (MADRS) score of 35 or more or by a Hamilton Depression Rating Scale (HAM-D) score of 25 or more.

The reduction or elimination of psychomotor retardation in a patient suffering from MDD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from MDD occurs not later than about 2 hours after the last administration of 5-MeO-DMT or a pharmaceutically acceptable salt thereof. The reduction or elimination of psychomotor retardation in a patient suffering from MDD preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from MDD, in particular of lassitude, is reflected by an improvement at least in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from MDD, in particular of lassitude, as reflected by an improvement in the score of the MADRS item “lassitude” occurs not later than about 2 hours after the last administration of 5-MeO-DMT or a pharmaceutically acceptable salt thereof. The reduction or elimination of psychomotor retardation in a patient suffering from MDD, in particular of lassitude, as reflected by an improvement in the score of the MADRS item “lassitude” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from MDD, in particular of psychomotor retardation, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from MDD, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from MDD, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of psychomotor retardation in a patient suffering from MDD, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering MDD, as reflected by at least an improvement in the score of the Salpêtrière Retardation Rating Scale (SRRS), occurs not later than about 2 hours after the last administration of 5-Me-ODMT or a pharmaceutically acceptable salt thereof. The reduction or elimination of psychomotor retardation in a patient suffering from MDD, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from MDD, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from MDD, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering from MDD, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to MDD. An improvement in psychomotor retardation will therefore also lead to an improvement of MDD. Since psychomotor retardation furthermore also affects other aspects of MDD, the inventors conclude that the improvement in psychomotor retardation, in particular the reduction or elimination of psychomotor retardation, will additionally contribute to an overall improvement of MDD.

An improvement of MDD in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of MDD in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of MDD in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients, suffering from MDD associated with psychomotor retardation, preferably using dosing schemes as described herein, without a significant risk of inducing mania or hypomania.

Preferably, the patient suffering from MDD associated with psychomotor retardation does not experience treatment-emergent mania or hypomania.

Bipolar Disorder (BD) is a mental health condition characterized by extreme mood swings that include emotional lows (major depressive episodes) and highs (manic or hypomanic episodes). Bipolar disorder is a recurrent chronic disorder that affects more than 1% of the world's population irrespective of ethnic origin or socioeconomic status.

BD is classified as Bipolar I Disorder if there has been at least one manic episode, with or without depressive episodes. It is classified as Bipolar II Disorder if there has been at least one hypomanic episode (but no full manic episodes) and one major depressive episode. If these symptoms are due to drugs or medical problems, they are not diagnosed as Bipolar Disorder.

The patient suffering from BD including Bipolar I Disorder and Bipolar II Disorder may suffer from a treatment resistant form of the disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of depression.

Psychomotor retardation is a core feature of bipolar depression and is one of the 9 symptoms listed in the DSM-V to identify a major depressive episode in the context of bipolar disorder.

A study comparing patients with bipolar depression to age-matched patients with unipolar depression found that bipolar disorder was associated with greater psychomotor retardation scores than unipolar depression at the same depression severity score.

Psychomotor retardation in a patient suffering from BD may be assessed as part of the determination of the HAM-D or the MADRS. Psychomotor retardation or at least aspects thereof may furthermore be assessed, for instance, by using the Salpêtrière Retardation Rating Scale (SRRS) or the Motor Agitation and Retardation Scale (MARS).

Patients suffering from Bipolar Disorder show characteristic aberrant intrinsic organization and interconnectivity of resting state networks.

In comparison with healthy controls, resting state functional magnetic resonance imaging studies demonstrated functional connectivity alterations of specific regions within and/or between the default mode network, the salience network and the central executive network.

Treating a patient suffering from BD, including a treatment resistant form of the disorder, and associated psychomotor retardation with 5-MeO-DMT or a pharmaceutically acceptable salt thereof reduces or eliminates the psychomotor retardation and leads to an improvement of BD.

Typically, the patient suffering from BD, whether diagnosed with bipolar II disorder or with bipolar I disorder, suffers from a current major depressive episode.

The severity of a current major depressive episode may be assessed using the Montgomery-Åsberg Depression Rating Scale (MADRS). The patient may have a total score of equal to or greater than 19, such as greater or equal than 24, in particular greater or equal than 37.

The reduction or elimination of psychomotor retardation in a patient suffering from BD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from BD occurs not later than about 2 hours after the last administration of 5-MeO-DMT or a pharmaceutically acceptable salt thereof. The reduction or elimination of psychomotor retardation in a patient suffering from BD preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from BD, in particular of lassitude, is reflected by an improvement at least in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Bipolar Disorder, in particular lassitude, as reflected by an improvement in the score of the MADRS item “lassitude” occurs not later than about 2 hours after the last administration of 5-MeO-DMT or a pharmaceutically acceptable salt thereof. The reduction or elimination of psychomotor retardation in a patient suffering from BD, as reflected by an improvement in the score of the MADRS item “lassitude” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from BD, in particular of retardation, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from BD, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from BD, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, a reduction or psychomotor retardation in a patient suffering from BD, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering BD, as reflected by at least an improvement in the score of the Salpêtrière Retardation Rating Scale (SRRS), occurs not later than about 2 hours after the last administration of 5-Me-ODMT or a pharmaceutically acceptable salt thereof. The reduction or elimination of psychomotor retardation in a patient suffering from BD, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, a reduction or psychomotor retardation in a patient suffering from BD, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering BD, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering from BD, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to BD. An improvement in psychomotor retardation will therefore also lead to an improvement of BD. Since psychomotor retardation furthermore also affects other aspects of BD, the inventors conclude that the improvement in psychomotor retardation will additionally contribute to an overall improvement of BD.

An improvement of the BD in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the BD in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the BD in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof. Compared to other psychedelics, like LSD, psylocibin or DMT, 5-MeO-DMT or a pharmaceutical acceptable salt thereof can be administered to patients suffering from Bipolar Disorder, such as Bipolar I Disorder and Bipolar II Disorder, associated with psychomotor retardation, preferably using dosing schemes as described herein, without a significant risk of inducing mania or hypomania.

Preferably, the patient suffering from Bipolar Disorder, such as Bipolar I Disorder and Bipolar II Disorder, associated with psychomotor retardation does not experience treatment-emergent mania or hypomania.

Postpartum Depression (PPD) is a debilitating mood disorder occurring during pregnancy or within 4 weeks following delivery. While over 50% of women may experience short-lasting low mood or tearfulness after childbirth, a subset of women may develop PPD. Epidemiological studies estimate that the prevalence rate of PPD is about 15%.

The patient suffering from PPD may suffer from a treatment resistant form of the disorder.

Postpartum depression is also defined as a major depressive episode within 4 weeks of delivery. As such, psychomotor retardation is a core feature of postpartum depression, and is one of the 9 symptoms listed in the DSM-V to identify a major depressive episode.

Psychomotor retardation in a patient suffering from PPD may be assessed as part of the determination of the HAM-D, or the MADRS. Psychomotor retardation or at least aspects thereof may furthermore be assessed, for instance, by the Salpêtrière Retardation Rating Scale (SRRS) or the Motor Agitation and Retardation Scale (MARS).

In patients with PPD, significant changes in neural activity in brain regions important for self-regulation, empathy, emotion, and cognition are observed. PPD is associated with dysfunctional connectivity of resting state networks, for instance, within and/or between the default mode network and frontoparietal network.

Treating a patient suffering from PPD, including a treatment resistant form of the disorder, and associated psychomotor retardation with 5-MeO-DMT or a pharmaceutically acceptable salt thereof reduces or eliminates psychomotor retardation and leads to an improvement of PPD.

A patient may suffer from moderate or severe PPD as indicated by a Montgomery-Åsberg Depression Rating Scale (MADRS) score of 20 or more or by a Hamilton Depression Rating Scale (HAM-D) score of 16 or more. It is further considered that the patient may suffer from severe PPD as indicated by a Montgomery-Åsberg Depression Rating Scale (MADRS) score of 35 or more or by a Hamilton Depression Rating Scale (HAM-D) score of 27 or more.

A patient treated according to the invention may have a Montgomery-Åsberg Depression Rating Scale (MADRS) score of 20 or more or a 17-item Hamilton Depression Rating Scale (HAM-D) score of 16 or more.

Further, a patient treated according to the invention may have a MADRS score of 28 or more or a HAM-D score of 22 or more.

Still further, a patient treated according to the invention may have a MADRS score of 35 or more or a HAM-D score of 25 or more.

The reduction or elimination of psychomotor retardation in a patient suffering from PPD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PPD occurs not later than about 2 hours after the last administration of 5-MeO-DMT or a pharmaceutically acceptable salt thereof. The reduction or elimination of psychomotor retardation in a patient suffering from PPD preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from PPD, in particular of lassitude, is reflected by an improvement at least in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PPD, in particular of lassitude, as reflected by an improvement in the score of the MADRS item “lassitude” occurs not later than about 2 hours after the last administration of 5-Me-ODMT or a pharmaceutically acceptable salt thereof. The reduction or elimination of psychomotor retardation in a patient suffering from PPD, in particular of lassitude, as reflected by an improvement in the score of the MADRS item “lassitude” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from PPD, in particular of retardation, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PPD, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from PPD, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of psychomotor retardation in a patient suffering from PPD, is reflected by at least an improvement in the score of the Salpêtrière Retardation Rating Scale about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering PPD, as reflected by at least an improvement in the score of the Salpêtrière Retardation Rating Scale, occurs not later than about 2 hours after the last administration of 5-MeO-DMT or a pharmaceutically acceptable salt thereof. The reduction or elimination of psychomotor retardation in a patient suffering PPD, as reflected by at least an improvement in the score of the Salpêtrière Retardation Rating Scale, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of psychomotor retardation in a patient suffering from PPD, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering PPD, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering PPD, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to PPD. An improvement in psychomotor retardation will therefore also lead to an improvement of PPD. Since psychomotor retardation furthermore also affects other aspects of PPD, the inventors conclude that the improvement in psychomotor retardation, will additionally contribute to an overall improvement of PPD.

An improvement of PPD in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of PPD in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of PPD in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Psychomotor retardation moreover compromises maternal functioning.

Maternal functioning can, for instance, be assessed using the Barkin Index of Maternal Functioning (BIMF). This index was designed to measure functioning in the year after childbirth. The BIMF is a 20-item self-report measure of functioning. Each item is assigned a score between 0 and 6 so that the maximum total score is 120. The higher the score, the better maternal functioning is rated.

The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, a score of 80 or below is considered herein as representing compromised maternal functioning, a score of 65 or below is considered herein as representing severely compromised maternal functioning.

The inventors have determined that increases in the score of the MADRS item “lassitude” have negative impacts on both aspects of maternal functioning (maternal competence relating to interactions with the infant(s) as well as maternal self-care).

Increased scores in the MADRS item “lassitude” impair self-care, psychological well-being and management. Conversely, improvements regarding this MADRS item lead to improvements in maternal functioning, in particular in the BIMF functional domains psychological wellbeing, social support and/or management.

The inventors conclude that 5-MeO-DMT can be used to treat PPD patients to achieve an improvement of psychomotor retardation, in particular a reduction or elimination of lassitude.

The inventors furthermore conclude that a reduction or elimination of lassitude by treating a PPD patient does not only lead to a reduction in the MADRS total score, but also to an improvement in maternal functioning, as reflected by an increase in the BIMF score.

The BIMF total score is improved by 10% or more, preferably by 20% or more.

The improvement of maternal functioning in a patient suffering from PPD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from PPD, as reflected by at least an improvement in the BIMF total score, occurs not later than about 6 days after the last administration of 5-MeO-DMT or a pharmaceutically acceptable salt thereof. The improvement of maternal functioning, as reflected by at least an improvement in the BIMF total score, preferably persists until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from PPD associated with psychomotor retardation, preferably using dosing schemes as described herein, without a significant risk of inducing mania or hypomania.

Preferably, the patient suffering from PPD associated with psychomotor retardation does not experience treatment-emergent mania or hypomania.

Seasonal Affective Disorder is a mood disorder with seasonal pattern, with symptoms often beginning in autumn and remitting in spring. Many people experience sadness, hopelessness, a loss of interest in activities, fatigue, and social withdrawal.

The patient suffering from Seasonal Affective Disorder may suffer from a treatment resistant form of the disorder.

Compared to controls, patients suffering from Seasonal Affective Disorder show significant season-independent impairments in tasks measuring working memory, cognitive processing speed and motor speed.

Sensorimotor impairment has been reported in depressed subjects compared to healthy controls.

Psychomotor symptoms associated with depressed mood are often thought to have an impact not only on the execution of motor responses, but also on the speed of thinking.

Seasonal Affective Disorder is associated with psychomotor retardation, because depressive episodes may meet the DSM-V criteria for a major depressive episode.

Psychomotor retardation in a patient suffering from Seasonal Affective Disorder may be assessed as part of the determination of the HAM-D, or the MADRS. Psychomotor retardation or at least aspects thereof may furthermore be assessed, for instance, the Salpêtrière Retardation Rating Scale (SRRS) or the Motor Agitation and Retardation Scale (MARS).

Resting state activity involved in sensorimotor, attention, and visual processing is altered in patients with seasonal affective disorder compared to healthy controls.

Treating a patient suffering from Seasonal Affective Disorder, including a treatment resistant form of the disorder, and associated psychomotor retardation with 5-MeO-DMT or a pharmaceutically acceptable salt thereof reduces or eliminates psychomotor retardation and leads to an improvement of the Seasonal Affective Disorder.

The reduction or elimination of psychomotor retardation in a patient suffering from Seasonal Affective Disorder is observed about 2 hours after the last administration of 5-Me-ODMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Seasonal Affective Disorder occurs not later than about 2 hours after the last administration of 5-MeO-DMT or a pharmaceutically acceptable salt thereof. The reduction or elimination of psychomotor retardation in a patient suffering from Seasonal Affective Disorder preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from Seasonal Affective Disorder, in particular of lassitude, is reflected by at least an improvement in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Seasonal Affective Disorder, in particular of lassitude, as reflected by an improvement in the score of the MADRS item “lassitude”, occurs not later than about 2 hours after the last administration of 5-MeO-DMT or a pharmaceutically acceptable salt thereof. The reduction or elimination of psychomotor retardation in a patient suffering from Seasonal Affective Disorder, as reflected by an improvement in the score of the MADRS item “lassitude”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from Seasonal Affective Disorder, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Seasonal Affective Disorder, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from Seasonal Affective Disorder, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, a reduction or psychomotor retardation in a patient suffering from Seasonal Affective Disorder, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from Seasonal Affective Disorder, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from Seasonal Affective Disorder, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, a reduction or psychomotor retardation in a patient suffering from Seasonal Affective Disorder, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from Seasonal Affective Disorder, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering from Seasonal Affective Disorder, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to Seasonal Affective Disorder. An improvement in psychomotor retardation will therefore also lead to an improvement of Seasonal Affective Disorder. Since psychomotor retardation furthermore also affects other aspects of Seasonal Affective Disorder, the inventors conclude that the improvement in psychomotor retardation will additionally contribute to an overall improvement of Seasonal Affective Disorder.

An improvement of Seasonal Affective Disorder in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Seasonal Affective Disorder in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of Seasonal Affective Disorder in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Seasonal Affective Disorder associated with psychomotor retardation, preferably using dosing schemes as described herein, without a significant risk of inducing mania or hypomania.

Preferably, the patient suffering from Seasonal Affective Disorder associated with psychomotor retardation does not experience treatment-emergent mania or hypomania.

### Persistent Depressive Disorder

Persistent Depressive Disorder, also referred to as dysthymia, is a chronic form of depression. It is diagnosed if depression is present for most of the day for the majority of days over at least a two-year period. Any symptom-free period is less than 2 months.

While depressed, two or more of the following must be present: 1. Hopelessness; 2. Energy low or fatigue; 3. Self-esteem is low; 4. Sleep decreased (insomnia) or increased (hypersomnia): 5. Appetite poor, or overeating; 6. Difficulty making decisions or poor concentration.

The patient suffering from Persistent Depressive Disorder may suffer from a treatment resistant form of the disorder.

According to the DSM-V, Persistent Depressive Disorder is characterized by criteria for a major depressive disorder which may be continuously present for 2 years.

Persistent Depressive Disorder is associated with psychomotor retardation, because depressive episodes may meet the DSM-V criteria for a major depressive episode.

Psychomotor retardation in a patient suffering from PDD may be assessed as part of the determination of the HAM-D, or the MADRS. Psychomotor retardation or at least aspects thereof may furthermore be assessed, for instance, by using the Salpêtrière Retardation Rating Scale (SRRS) or the Motor Agitation and Retardation Scale (MARS).

In patients suffering from Persistent Depressive Disorder altered functional connectivity is observed within and/or between several brain regions implicated in processing, regulation, emotional memory; cognitive processes related to rumination; impaired concentration and physiological arousal.

Dysfunctional connectivity is observed within and between the DMN, salience network, executive control network and limbic network. Functional connectivity differs significantly from that observed in healthy controls.

Treating a patient suffering from PDD, including a treatment resistant form of the disorder, and associated psychomotor retardation with 5-MeO-DMT or a pharmaceutically acceptable salt thereof reduces or eliminates psychomotor retardation and leads to an improvement of PDD.

The reduction or elimination of psychomotor retardation in a patient suffering from PDD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PDD occurs not later than about 2 hours after the last administration of 5-MeO-DMT or a pharmaceutically acceptable salt thereof. The reduction or elimination of psychomotor retardation in a patient suffering from PDD preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from PDD, in particular of lassitude, is reflected by an improvement at least in the score of the MADRS item “lassitude” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PDD, in particular of lassitude, as reflected by an improvement in the score of the MADRS item “lassitude” occurs not later than about 2 hours after the last administration of 5-Me-ODMT or a pharmaceutically acceptable salt thereof. The reduction or elimination of psychomotor retardation in a patient suffering from PDD, in particular of lassitude, as reflected by an improvement in the score of the MADRS item “lassitude”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from PDD, in particular of psychomotor retardation, is reflected by at least an improvement in the score of the HAM-D item retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PDD, in particular of retardation, as reflected by an improvement in the score of the HAM-D item retardation, occurs not later than about 2 hours after the last administration of 5-MeO-DMT or a pharmaceutically acceptable salt thereof. The reduction or elimination of psychomotor retardation in a patient suffering from PDD, in particular of retardation, as reflected by an improvement in the score of the HAM-D item retardation, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of psychomotor retardation in a patient suffering from PDD, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering PDD, as reflected by at least an improvement in the score of the Salpêtrière Retardation Rating Scale (SRRS), occurs not later than about 2 hours after the last administration of 5-Me-ODMT or a pharmaceutically acceptable salt thereof. The reduction or elimination of psychomotor retardation in a patient suffering from PDD, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from PDD, is reflected by at least an improvement in the average score of the Motor Agitation and Retardation Scale (MARS) subscale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PDD, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering from PDD, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to PDD. An improvement in psychomotor retardation will therefore also lead to an improvement of PDD. Since psychomotor retardation furthermore also affects other aspects of PDD, the inventors conclude that the improvement in psychomotor retardation, in particular the reduction or elimination of psychomotor retardation, will additionally contribute to an overall improvement of PDD.

An improvement of PDD in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of PDD in a patient also suffering from associated psychomotor retardation, as reflected by a reduction in the CGI-S score, occurs not later than about 2 hours after the last administration of 5-MeO-DMT or a pharmaceutically acceptable salt thereof.

An improvement of PDD in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Persistent Depressive Disorder associated with psychomotor retardation, preferably using dosing schemes as described herein, without a significant risk of inducing mania or hypomania.

Preferably, the patient suffering from Persistent Depressive Disorder associated with psychomotor retardation does not experience treatment-emergent mania or hypomania.

### Mental and Behavioural Disorders due to Psychoactive Substance Use

A Substance Use Disorder (SUD) is a mental disorder that affects a person's behaviour, leading to a person's inability to control their use of substances such as legal or illegal drugs, alcohol, or medications. Symptoms can range from moderate to severe, with addiction being the most severe form of SUDs.

A patient suffering from SUD may suffer from a treatment resistant form of the disorder.

Psychomotor retardation occurs in patients suffering from certain mental and behavioural disorders due to psychoactive substance use.

Psychomotor retardation has been noted in methamphetamine withdrawal, with one study showing female patients with methamphetamine use disorder are more likely to experience psychomotor retardation than male users.

A large study of frequent cannabis users found psychomotor retardation to be amongst the most prevalent symptoms of cannabis withdrawal, although only at 1% lifetime prevalence.

Resting state functional connectivity (rsFC) was found to be altered not only in patients with psychomotor retardation, but also in patients with SUD.

Psychomotor retardation or at least aspects thereof may also be assessed, for instance, by using the Salpêtrière Retardation Rating Scale (SRRS).

Treating a patient suffering from SUD, including a treatment resistant form of the disorder, and associated psychomotor retardation with 5-MeO-DMT or a pharmaceutically acceptable salt thereof reduces or eliminates the psychomotor retardation and leads to an improvement of SUD.

The reduction or elimination of psychomotor retardation in a patient suffering from SUD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from SUD occurs not later than about 2 hours after the last administration of 5-MeO-DMT or a pharmaceutically acceptable salt thereof. The reduction or elimination of psychomotor retardation in a patient suffering from SUD preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from SUD is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from SUD, as reflected by at least an improvement in the score of the Salpêtrière Retardation Rating Scale (SRRS), occurs not later than about 2 hours after the last administration of 5-Me-ODMT or a pharmaceutically acceptable salt thereof. The reduction or elimination of psychomotor retardation in a patient suffering from SUD, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from SUD. An improvement in psychomotor retardation will therefore also lead to an improvement of SUD. Since psychomotor retardation furthermore also affects other aspects of SUD, the inventors conclude that the improvement in psychomotor retardation will additionally contribute to an overall improvement of Substance Use Disorder.

An improvement of the SUD in a patient suffering from psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the SUD in a patient suffering from psychomotor retardation, as reflected by a reduction in the CGI-S score, occurs not later than about 2 hours after the last administration of 5-MeO-DMT or a pharmaceutically acceptable salt thereof. An improvement in psychomotor retardation in a patient also suffering from SUD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Psychotic Disorders

Psychotic Disorders are severe mental disorders that cause abnormal thinking and perceptions. Psychotic disorders are characterised by significant impairments in reality testing and alterations in behaviour manifest in positive symptoms such as persistent delusions, persistent hallucinations, disorganised thinking (typically manifest as disorganised speech), grossly disorganised behaviour, and experiences of passivity and control, negative symptoms such as blunted or flat affect and avolition, and psychomotor disturbances.

A patient suffering from a Psychotic Disorder may suffer from a treatment resistant form of the disorder.

The DSM-V describes symptoms of psychomotor retardation such as lack of verbal and motor responses a core feature that define psychotic disorders. Schizophrenia is an example of a primary psychotic disorder in which psychomotor retardation is a prominent feature.

Psychomotor retardation is also noted in other psychotic disorders such as schizoaffective disorder.

Psychomotor retardation or at least aspects thereof may furthermore be assessed, for instance, the Salpêtrière Retardation Rating Scale (SRRS).

Brain imaging of patients suffering from psychosis by functional magnetic resonance imaging of brain resting state networks, reveals profound alterations in distinct regions within and/or between the central executive network, the default mode network and the salience network. Even in patient populations at risk for psychosis, alterations in resting state networks can be identified.

Treating a patient suffering from a Psychotic Disorder, including a treatment resistant from of the disorder, and associated psychomotor retardation with 5-MeO-DMT or a pharmaceutically acceptable salt thereof reduces or eliminates the psychomotor retardation and leads to an improvement of the Psychotic Disorder.

The reduction or elimination of psychomotor retardation in a patient suffering from a Psychotic Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from a Psychotic Disorder occurs not later than about 2 hours after the last administration of 5-Me-ODMT or a pharmaceutically acceptable salt thereof. The reduction or elimination of psychomotor retardation in a patient suffering from a Psychotic Disorder preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from a Psychotic Disorder is reflected by at least an improvement in the score of the Salpêtrière Retardation Rating Scale (SRRS) about 2 hours after the last administration of 5-Me-ODMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from a Psychotic Disorder, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from a Psychotic Disorder, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, the occurrence of psychomotor retardation is an important disease aspect in patients suffering from a Psychotic Disorder. An improvement in psychomotor retardation will therefore also lead to an improvement of the Psychotic Disorder. Since psychomotor retardation furthermore also affects other aspects of a Psychotic Disorder, the inventors conclude that the improvement in psychomotor retardation will additionally contribute to an overall improvement of the Psychotic Disorder.

An improvement of a Psychotic Disorder in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of a Psychotic Disorder in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the Psychotic Disorder in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from a Psychotic Disorder characterised by mania or hypomania and associated with psychomotor retardation, preferably using dosing schemes as described herein, without a significant risk of inducing mania or hypomania.

Preferably, the patient suffering from a Psychotic Disorder characterised by mania or hypomania and associated with psychomotor retardation does not experience treatment-emergent mania or hypomania.

Schizophrenia is a severe mental health condition characterised by disturbances in multiple mental modalities, including perception, self-experience, volition, affect and behaviour. Psychomotor disturbances, including catatonia, may be present.

A patient suffering from Schizophrenia may suffer from a treatment resistant form of the disorder.

Psychomotor retardation and slowing is also a core feature of Schizophrenia and classed as one of the ‘negative symptoms’ that characterises the disease. Manifestation can range from slowing of movements to severe catatonia.

There is evidence to suggest that psychomotor retardation is linked to more severe disease as well as reduced semantic and episodic memory in schizophrenia.

Abnormal resting state functional connectivity, particularly within and/or between the default mode network, the frontoparietal network and the salience network is reported in individuals with Schizophrenia.

Psychomotor retardation, or at least aspects thereof may be assessed, for instance, by the Salpêtrière Retardation Rating Scale (SRRS).

Treating a patient suffering from Schizophrenia, including a treatment resistant from of the disorder, and associated psychomotor retardation with 5-MeO-DMT or a pharmaceutically acceptable salt thereof reduces or eliminates the psychomotor retardation and leads to an improvement of Schizophrenia.

The reduction or elimination of psychomotor retardation in a patient suffering from Schizophrenia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Schizophrenia occurs not later than about 2 hours after the last administration of 5-MeO-DMT or a pharmaceutically acceptable salt thereof. The reduction or elimination of psychomotor retardation in a patient suffering from Schizophrenia preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from Schizophrenia, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from Schizophrenia, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from Schizophrenia, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to Schizophrenia. An improvement in psychomotor retardation will therefore also lead to an improvement of the Schizophrenia. Since psychomotor retardation furthermore also affects other aspects of Schizophrenia, the inventors conclude that the improvement in psychomotor retardation will additionally contribute to an overall improvement of Schizophrenia.

An improvement of Schizophrenia in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Schizophrenia in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of Schizophrenia in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Schizophrenia associated with psychomotor retardation, preferably using dosing schemes as described herein, without a significant risk of inducing mania or hypomania.

Preferably, the patient suffering from Schizophrenia associated with psychomotor retardation does not experience treatment-emergent mania or hypomania.

### Dementia

Dementia is caused by abnormal brain changes which trigger a decline in cognitive abilities and also affect behaviour, feelings and relationships. The condition is generally characterized by a loss of memory, language, problem-solving and other thinking abilities that are severe enough to interfere with daily life.

Psychomotor slowing is an important feature in dementia syndromes.

Psychomotor retardation, or at least aspects thereof may be assessed, for instance, by the Salpêtrière Retardation Rating Scale (SRRS).

Dementia affects various functional and structural connectivity networks in the brain, as can be shown by magnetic resonance imaging studies. Alterations within and/or between the default mode network (DMN), the salience network, and the central executive network (CEN) are observed.

Treating a patient suffering from Dementia and associated psychomotor retardation with 5-MeO-DMT or a pharmaceutically acceptable salt thereof reduces or eliminates the psychomotor retardation and leads to an improvement of Dementia.

The reduction or elimination of psychomotor retardation in a patient suffering from Dementia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Dementia occurs not later than about 2 hours after the last administration of 5-MeO-DMT or a pharmaceutically acceptable salt thereof. The reduction or elimination of psychomotor retardation in a patient suffering from Dementia preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from Dementia, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from Dementia, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from Dementia, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation occurs in patients suffering from Dementia. An improvement in psychomotor retardation will therefore also lead to an improvement of the Dementia. Since psychomotor retardation furthermore also affects other aspects of Dementia, the inventors conclude that the improvement in psychomotor retardation will additionally contribute to an overall improvement of the Dementia.

An improvement of the Dementia in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Dementia in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the Dementia in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Alzheimer's Dementia (AD) is a highly prevalent neurodegenerative disorder with an insidious onset and gradual progression of cognitive deficits. The percentage of neurocognitive disorders attributable to Alzheimer's disease ranges from about 60% to over 90%, depending on the setting and diagnostic criteria. Decline in learning and memory is an early and predominant feature, and the decline is progressive.

Psychomotor slowing is an important feature in dementia syndromes such as Alzheimer's disease. The pathological drivers for this may be slower perceptuomotor and decision processes.

A study of 148 patients with mild cognitive impairment and aged matched controls found that presence of psychomotor retardation significantly increased the risk of conversion to Alzheimer's dementia.

Psychomotor retardation, or at least aspects thereof may be assessed, for instance, by the Salpêtrière Retardation Rating Scale (SRRS).

AD affects various functional and structural connectivity networks in the brain which are associated with the topography, clinical phenotype, and severity of the disease. Magnetic resonance imaging studies have demonstrated successive structural and functional disconnection among brain regions supporting the idea that AD is a disconnection syndrome. In particular, alterations within and/or between the default mode network (DMN), salience network, and central executive network (CEN) are observed both in patients with AD and individuals who were at high risk for developing AD.

Therefore, influencing those networks by a therapy according to the invention will lead to an improvement of the psychomotor retardation, and also to an improvement of the symptoms of AD.

Treating a patient suffering from AD and associated psychomotor retardation with 5-MeO-DMT or a pharmaceutically acceptable salt thereof reduces or eliminates the psychomotor retardation and leads to an improvement of Alzheimer's Dementia.

The reduction or elimination of psychomotor retardation in a patient suffering from AD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from AD occurs not later than about 2 hours after the last administration of 5-MeO-DMT or a pharmaceutically acceptable salt thereof. The reduction or elimination of psychomotor retardation in a patient suffering from AD preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from AD, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering from AD, as reflected by at least an improvement in the score of the Salpêtrière Retardation Rating Scale (SRRS), occurs not later than about 2 hours after the last administration of 5-Me-ODMT or a pharmaceutically acceptable salt thereof. The reduction or elimination of psychomotor retardation in a patient suffering from AD, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from AD. An improvement in psychomotor retardation will therefore also lead to an improvement of the AD. Since psychomotor retardation furthermore also affects other aspects of AD, the inventors conclude that the improvement in psychomotor retardation will additionally contribute to an overall improvement of the AD.

An improvement of the AD in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the AD in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the AD in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Dementia with Lewy Bodies Dementia (DLB) is a type of dementia characterized by changes in sleep, behaviour, cognition, movement, and regulation of automatic bodily functions.

Psychomotor slowing plays an important role in Dementia with Lewy Bodies (DLB). In DLB, psychomotor slowing may be underpinned by visual and attention disorders which also contribute to hallucinations. DLB is characterised by worse psychomotor impairments than Alzheimer's disease.

Psychomotor retardation, or at least aspects thereof may be assessed, for instance, by the Salpêtrière Retardation Rating Scale (SRRS).

In patients suffering from DLB, resting state network (RSN) alterations can be identified. Functional MRI studies show changes in functional connectivity within and/or between the default mode network, salience network, executive network and basal ganglia/limbic network.

Treating a patient suffering from DLB and associated psychomotor retardation with 5-MeO-DMT or a pharmaceutically acceptable salt thereof reduces or eliminates the psychomotor retardation and leads to an improvement of DLB.

The reduction or elimination of psychomotor retardation in a patient suffering from DLB is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from DLB occurs not later than about 2 hours after the last administration of 5-MeO-DMT or a pharmaceutically acceptable salt thereof. The reduction or elimination of psychomotor retardation in a patient suffering from DLB preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from DLB is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from DLB, as reflected by an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from DLB, as reflected by an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from DLB. An improvement in psychomotor retardation will therefore also lead to an improvement of the DLB. Since psychomotor retardation furthermore also affects other aspects of DLB, the inventors conclude that the improvement in psychomotor retardation will additionally contribute to an overall improvement of the DLB.

An improvement of the DLB in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the DLB in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the DLB in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Vascular Dementia is a common type of dementia characterized by problems with reasoning, planning, judgment, memory and other thought processes. These problems are caused by brain damage from impaired blood flow to the brain.

Psychomotor retardation is highlighted as a clinical feature consistent with a diagnosis of probable vascular dementia as per the NINDS-AIREN (National Institute of Neurological Disorders and Stroke/Association Internationale pour la Recherche et l'Enseignement en Neurosciences research) criteria.

Psychomotor retardation is especially frequent when vascular dementia affects the subcortical territory.

Psychomotor retardation or at least aspects thereof may be assessed, for instance, by using the Salpêtrière Retardation Rating Scale (SRRS).

Functional magnetic resonance imaging reveals altered functional connectivity of resting state networks after a cerebrovascular insult. Patients suffering from major or mild vascular neurocognitive disorder show an abnormal functional connectivity within the DMN and other resting state networks.

Treating a patient suffering from Vascular Dementia and associated psychomotor retardation with 5-MeO-DMT or a pharmaceutically acceptable salt thereof reduces or eliminates the psychomotor retardation and leads to an improvement of Vascular Dementia.

The reduction or elimination of psychomotor retardation in a patient suffering from Vascular Dementia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Vascular Dementia occurs not later than about 2 hours after the last administration of 5-MeO-DMT or a pharmaceutically acceptable salt thereof. The reduction or elimination of psychomotor retardation in a patient suffering from Vascular Dementia preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from Vascular Dementia is reflected by at least an improvement in the score of the Salpêtrière Retardation Rating Scale (SRRS) about 2 hours after the last administration of 5-Me-ODMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Vascular Dementia, as reflected by an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from Vascular Dementia, as reflected by an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from Vascular Dementia. An improvement in psychomotor retardation will therefore also lead to an improvement of the Vascular Dementia. Since psychomotor retardation furthermore also affects other aspects of Vascular Dementia, the inventors conclude that the improvement in psychomotor retardation will additionally contribute to an overall improvement of the Vascular Dementia.

An improvement of the Vascular Dementia in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Vascular Dementia in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of the Vascular Dementia in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Parkinson's Disease Dementia is characterized by changes in thinking and behaviour in a patient with a diagnosis of Parkinson's disease, an illness characterized by gradually progressive problems with movement, most commonly involving slowing of movements, a tremor present at rest, and walking instability which can cause falls.

Parkinson's Disease Dementia is also associated with psychomotor retardation.

Studies have shown the presence of a functional reorganization of resting state networks which are critical to generate and maintain an efficient behavioural and cognitive performance in patients suffering from Parkinson's Disease Dementia. Levodopa, which improves the clinical status of Parkinson patients, has the potential to influence functional connectivity of resting state networks in Parkinson patients.

Treating a patient suffering from Parkinson's Disease Dementia and associated psychomotor retardation with 5-MeO-DMT or a pharmaceutically acceptable salt thereof reduces or eliminates psychomotor retardation and leads to an improvement of the Parkinson's Disease Dementia.

The reduction or elimination of psychomotor retardation in a patient suffering from Parkinson's Disease Dementia is observed on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Parkinson's Disease Dementia occurs not later than about 24 hours after the last administration of 5-MeO-DMT or a pharmaceutically acceptable salt thereof. The reduction or elimination of psychomotor retardation in a patient suffering from Parkinson's Disease Dementia preferably persists until at least 6 days; in particular until at least 14 days; more preferably until at least 28 days after the last administration of 5-MeO-DMT or a pharmaceutically acceptable salt thereof.

An improvement in psychomotor retardation in a patient suffering from Parkinson's Disease Dementia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in psychomotor retardation in a patient suffering from Parkinson's Disease Dementia, as reflected by a reduction in the CGI-S score, occurs not later than about 24 hours after the last administration of 5-MeO-DMT or a pharmaceutically acceptable salt thereof. An improvement in psychomotor retardation in a patient suffering from Parkinson's Disease Dementia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

A reduction or elimination of psychomotor retardation in a patient suffering from Parkinson's Disease Dementia is reflected by an improvement in the score of the Salpêtrière Retardation Rating Scale (SRRS) on day 1, for instance, after about 24 hours; on day 7; on day 14 and/or on day 28 after the last administration of 5-MeO-DMT or a pharmaceutically acceptable salt thereof.

A reduction or elimination of psychomotor retardation in a patient suffering from Parkinson's Disease Dementia, as reflected by an improvement in the score of the Salpêtrière Retardation Rating Scale (SRRS), occurs not later than about 24 hours after the last administration of 5-MeO-DMT or a pharmaceutically acceptable salt thereof. The reduction or elimination of psychomotor retardation in a patient suffering from PDD, as reflected by an improvement in the of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect of Parkinson's Disease Dementia. An improvement in psychomotor retardation will therefore also lead to an improvement of the Parkinson's Disease Dementia. Since psychomotor retardation furthermore also affects other aspects of Parkinson's Disease Dementia, the inventors conclude that the improvement in psychomotor retardation will additionally contribute to an overall improvement of the Parkinson's Disease Dementia.

An improvement of the Parkinson's Disease Dementia in a patient also suffering from associated psychomotor retardation, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Parkinson's Disease Dementia in a patient also suffering from associated psychomotor retardation, as reflected by a reduction in the CGI-S score, occurs not later than about 24 hours after the last administration of 5-MeO-DMT or a pharmaceutically acceptable salt thereof. An improvement of the Parkinson's Disease Dementia in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Parkinson's Disease

Parkinson's Disease (PD) is an illness characterized by gradually progressive problems with movement, most commonly involving slowing of movements, a tremor present at rest, and walking instability which can cause falls.

Psychomotor retardation is a cardinal feature of subcortical disorders such as Parkinson's Disease (PD).

Psychomotor retardation or at least aspects thereof may be assessed, for instance, by using the Salpêtrière Retardation Rating Scale (SRRS) or the Digit Symbol Substitution Test (DSST).

Analysis of resting state networks using functional magnetic resonance imaging show that Parkinson's disease related cognitive deficits are associated with patterns of altered connectivity within and/or between resting-state functional connectivity networks, such as the default mode network, dorsal attention networks or frontoparietal network.

Treating a patient suffering from PD and associated psychomotor retardation with 5-MeO-DMT or a pharmaceutically acceptable salt thereof reduces or eliminates the psychomotor retardation and leads to an improvement of PD.

The reduction or elimination of psychomotor retardation in a patient suffering from PD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PD occurs not later than about 2 hours after the last administration of 5-MeO-DMT or a pharmaceutically acceptable salt thereof. The reduction or elimination of psychomotor retardation in a patient suffering from PD preferably persists until at least 6 days; in particular until at least 14 days; more preferably until at least 28 days after the last administration of 5-MeO-DMT or a pharmaceutically acceptable salt thereof.

The reduction or elimination of psychomotor retardation in a patient suffering from PD is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PD, as reflected by an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from PD, as reflected by an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from PD is reflected by at least an improvement in the score of the Digit Symbol Substitution Test (DSST) about 2 hours after the last administration of 5-Me-ODMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from PD, as reflected by an improvement in the score of the Digit Symbol Substitution Test (DSST), occurs not later than about 2 hours after the last administration of 5-MeO-DMT or a pharmaceutically acceptable salt thereof. The reduction or elimination of psychomotor retardation in a patient suffering from PD, as reflected by an improvement in the score of the Digit Symbol Substitution Test (DSST), preferably persists until at least 6 days; in particular until at least 14 days; more preferably until at least 28 days after the last administration of 5-MeO-DMT or a pharmaceutically acceptable salt thereof.

As indicated above, psychomotor retardation is an important aspect in patients suffering from PD. An improvement in psychomotor retardation will therefore also lead to an improvement of the PD. Since psychomotor retardation furthermore also affects other aspects of PD, the inventors conclude that the improvement in psychomotor retardation will additionally contribute to an overall improvement of the PD.

An improvement in psychomotor retardation in a patient suffering from PD as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement in psychomotor retardation in a patient suffering from PD, as reflected by a reduction in the CGI-S score, occurs not later than about 2 hours after the last administration of 5-MeO-DMT or a pharmaceutically acceptable salt thereof. An improvement in psychomotor retardation in a patient also suffering from PD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Chronic Fatigue Syndrome

Fatigue describes a feeling of exhaustion, lethargy, and decreased energy. Fatigue is experienced as a weakening or depletion of one's physical or mental resources. Even though considered as normal following a period of exertion, mental or physical, fatigue may occur in the absence of such exertion as a symptom of health conditions.

Chronic fatigue exacerbated by activity is moreover a prominent symptom in Chronic Fatigue Syndrome. In this disorder, severe fatigue is accompanied by neurocognitive, autonomic, and immunological symptoms.

A variety of abnormalities in normal sleep patterns, which may act as perpetuating factors, have been reported in Chronic Fatigue Syndrome patients.

Symptoms of Chronic Fatigue Syndrome also include psychomotor slowing.

Patients suffering from Chronic Fatigue Syndrome show abnormal resting state functional connectivity, significantly correlated with the severity of their chronic fatigue.

Treating a patient suffering from Chronic Fatigue Syndrome, including a treatment resistant form of the disorder, and associated psychomotor retardation with 5-MeO-DMT or a pharmaceutically acceptable salt thereof reduces or eliminates psychomotor retardation and leads to an improvement of Chronic Fatigue Syndrome.

The reduction or elimination of psychomotor retardation in a patient suffering from Chronic Fatigue Syndrome is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Chronic Fatigue Syndrome occurs not later than about 2 hours after the last administration of 5-MeO-DMT or a pharmaceutically acceptable salt thereof. The reduction or elimination of psychomotor retardation in a patient suffering from Chronic Fatigue Syndrome preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of psychomotor retardation in a patient suffering from Chronic Fatigue Syndrome, is reflected by at least an improvement in the score of the Salpêtrière Retardation Rating Scale (SRRS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of psychomotor retardation in a patient suffering Chronic Fatigue Syndrome, as reflected by at least an improvement in the score of the Salpêtrière Retardation Rating Scale (SRRS), occurs not later than about 2 hours after the last administration of 5-MeO-DMT or a pharmaceutically acceptable salt thereof. The reduction or elimination of psychomotor retardation in a patient suffering from Chronic Fatigue Syndrome, as reflected by at least an improvement in the score of the Salpêtrière Retardation Rating Scale (SRRS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation in a patient suffering from Chronic Fatigue Syndrome, is reflected by at least an improvement in the average score of the Motor Agitation and Retardation Scale (MARS) sub-scale related to motor retardation about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of psychomotor retardation in a patient suffering from Chronic Fatigue Syndrome, as reflected by at least an improvement in the average score of the Motor Agitation and Retardation Scale (MARS) subscale related to motor retardation, occurs not later than about 2 hours after the last administration of 5-MeO-DMT or a pharmaceutically acceptable salt thereof. The reduction or elimination of psychomotor retardation in a patient suffering from Chronic Fatigue Syndrome, as reflected by at least an improvement in the average score of the Motor Agitation and Retardation Scale (MARS) subscale related to motor retardation, preferably persists until at least 6 days; in particular until at least 14 days; more preferably until at least 28 days after the last administration of 5-MeO-DMT or a pharmaceutically acceptable salt thereof.

As indicated above, psychomotor retardation is closely linked to Chronic Fatigue Syndrome. An improvement in psychomotor retardation will therefore also lead to an improvement of Chronic Fatigue Syndrome. Since psychomotor retardation furthermore also affects other aspects of Chronic Fatigue Syndrome, the inventors conclude that the improvement in psychomotor retardation, in particular the reduction or elimination of psychomotor retardation, will additionally contribute to an overall improvement of Chronic Fatigue Syndrome.

An improvement of Chronic Fatigue Syndrome in a patient also suffering from associated psychomotor retardation, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Chronic Fatigue Syndrome in a patient also suffering from associated psychomotor retardation, as reflected by a reduction in the CGI-S score, occurs not later than about 2 hours after the last administration of 5-MeO-DMT or a pharmaceutically acceptable salt thereof. An improvement of Chronic Fatigue Syndrome in a patient also suffering from associated psychomotor retardation, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### EXAMPLES

The following Examples are listed to aid understanding of the invention and are not intended and should not be construed to limit in any way the invention set forth in the claims which follow thereafter.

### Example 1-5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.

Step 2: 200 μl of the solution is transferred to a dosing capsule containing the drip pad (Storz & Bickel, Germany), and then the dosing capsule is closed with its lid.

Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.

Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.

Step 5: The balloon is then disconnected from the filling chamber, which automatically closes the valve. After attachment of the mouthpiece to the balloon, the aerosol is ready for immediate administration to the volunteer or patient.

Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1 M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5° 2·±0.2° 2·; 16.7° 2·±0.2° 2·; 17.02·±0.2° 2·; 20.6° 2·±0.2° 2·; 20.7° 2·±0.2° 2·; 21.4° 2·±0.2° 2·; 24.2° 2·±0.2° 2·; 24.82·±0.2° 2·; 25.3° 2·±0.2° 2·; 27.4° 2·±0.2° 2·; measured using Cu K· radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

### Binding to 5-HT1A Receptors in Post-Mortem Human Hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂ and 0.1% ascorbic acid. The assay buffer consisted of 50 mM Tris, pH 7.7, 4 mM CaCl₂, 0.1% ascorbic acid and 10 μM Pargyline.

For saturation binding analysis, hippocampal membranes (400 μl; equivalent 1.25 mg wet weight tissue/tube) was incubated with 50 μl of 0.075-9.6 nM [³H]8-OH-DPAT and either 50 μl of assay buffer (total binding) or 50 μl of 1 μM WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H]8-OH-DPAT and either 50 μl of assay buffer (total binding) or 50 μl of 1 ·M WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester. Filters were rapidly washed with ice-cold wash buffer (wash setting 0,9,9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

### Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 pI; equivalent to 4 mg wet weight of tissue/tube) were incubated with 50 μl of 0.00625-0.8 nM [³H]MDL-100,907 and either 50 μl of assay buffer or 50 μl of 10 ·M ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [3H]MDL-100,907 and either 50 μl of assay buffer (total binding) or 50 μl of 10 ·M ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

### Results

The dissociation constant (K_{d} value) of [³H]8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H]MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 pg/plate (the maximum recommended concentration according to current regulatory guidelines), in the absence and in the presence of a rat liver metabolic activation system (S-9).

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 1 below shows median percentage plasma concentrations relative to Cmax as determined for the time points indicated.

| time | | Median percentage plasma concentration relative to Cmax | | | | | | | | | | | | | |
|---|---|---|---|---|---|---|---|---|---|---|---|---|---|---|---|
| (min) | | 1 | 2 | 4 | 7 | 10 | 15 | 20 | 30 | 45 | 60 | 90 | 120 | 180 | 240 |
| 6 | mg | 96 | 98 | 83 | 56 | 40 | 22 | 16 | 10 | 8 | 4 | 2 | 1 | 0 | N.A. |
| 12 | mg | 100 | 79 | 59 | 30 | 22 | 12 | 8 | 5 | 3 | 2 | 0 | 0 | 0 | 0 |
| 18 | mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine. Only in few samples, concentrations were above the lower level of quantification (LLOQ) (25 pg/ml). From 15 min onwards, the bufotenine concentration was always below the LLOQ.

Substantially similar observations were made when subjects receiving an up-titration scheme were included.

### Example 7—Clinical Trial in Patients Suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intrapatient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 2-A
Scores recorded against relevant MADRS and BPRS items for patients assigned to
intra-day individualised dosing regimen (IDR). The item scores represent the sum
of the individual patient scores for all patients (n = 8) in the IDR group.
Assessment 2 hours (MADRS) or 3 hours (BPRS) after administration of the last dose.

| | | Baseline | 2 / 3 | Improvement | Percentage |
|---|---|---|---|---|---|
| Scale item | Scale | IDR | hours | 2 / 3 hours | improvement |
| Concentration | MADRS | 30 | 11 | 19 | 63.33% |
| Difficulties | | | | | |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 2-B
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 1.

| | | Baseline | | Improvement | Percentage |
|---|---|---|---|---|---|
| Scale item | Scale | IDR | Day 1 | at Day 1 | improvement |
| Reduced Sleep | MADRS | 25 | 12 | 13 | 52.00% |
| Concentration | MADRS | 30 | 1 | 29 | 96.67% |
| Difficulties | | | | | |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic Thoughts | MADRS | 28 | 4 | 24 | 85.71% |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional Withdrawal | BPRS | 13 | 8 | 5 | 38.46% |
| Blunted Affect | BPRS | 15 | 8 | 7 | 46.67% |

TABLE 2-C
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 7.

| | | Baseline | | Improvement | Percentage |
|---|---|---|---|---|---|
| Scale item | Scale | IDR | Day 7 | at Day 7 | improvement |
| Reduced Sleep | MADRS | 25 | 9 | 16 | 64.00% |
| Concentration | MADRS | 30 | 9 | 21 | 70.00% |
| Difficulties | | | | | |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt Feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional Withdrawal | BPRS | 13 | 8 | 5 | 38.46% |
| Blunted Affect | BPRS | 15 | 8 | 7 | 46.67% |

TABLE 3-A
Scores recorded against relevant MADRS and BPRS items for patients assigned
to 12 mg dosing regimen. The item scores represent the sum of the individual
patient scores for all patients (n = 4) in the 12 mg group. Assessment
2 hours (MADRS) or 3 hours (BPRS) after administration of the dose.

| | | | | Improvement | |
|---|---|---|---|---|---|
| | | Baseline | 2 / 3 | after 2 / 3 | Percentage |
| Scale item | Scale | 12 mg | hours | hours | improvement |
| Concentration | MADRS | 16 | 7 | 9 | 56.25% |
| Difficulties | | | | | |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic Thoughts | MADRS | 16 | 8 | 8 | 50.00% |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional Withdrawal | BPRS | 13 | 11 | 2 | 15.38% |
| Blunted Affect | BPRS | 11 | 8 | 3 | 27.27% |

TABLE 3-B
Scores recorded against relevant MADRS and BPRS items for patients assigned to
12 mg dosing regimen. The item scores represent the sum of the individual patient
scores for all patients (n = 4) in the 12 mg group. Assessment on day 1.

| | | Baseline | | Improvement | Percentage |
|---|---|---|---|---|---|
| Scale item | Scale | 12 mg | Day 1 | at Day 1 | improvement |
| Reduced Sleep | MADRS | 12 | 10 | 2 | 16.67% |
| Concentration | MADRS | 16 | 2 | 14 | 87.50% |
| Difficulties | | | | | |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic Thoughts | MADRS | 16 | 7 | 9 | 56.25% |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 8 | 5 | 38.46% |
| Blunted Affect | BPRS | 11 | 6 | 5 | 45.45% |

TABLE 3-C
Scores recorded against relevant MADRS and BPRS items for patients assigned to
12 mg dosing regimen. The item scores represent the sum of the individual patient
scores for all patients (n = 4) in the 12 mg group. Assessment on day 7.

| | | Baseline | | Improvement | Percentage |
|---|---|---|---|---|---|
| Scale item | Scale | 12 mg | Day 7 | at Day 7 | improvement |
| Reduced Sleep | MADRS | 12 | 6 | 6 | 50.00% |
| Concentration | MADRS | 16 | 3 | 13 | 81.25% |
| Difficulties | | | | | |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic Thoughts | MADRS | 16 | 8 | 8 | 50.00% |
| Suicidal Thoughts | MADRS | 8 | 7 | 1 | 12.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 6 | 7 | 53.85% |
| Blunted Affect | BPRS | 11 | 5 | 6 | 54.55% |

### Example 8—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0: 6 mg, 12 mg, and 18 mg.
1. All patients will receive an initial dose of 6 mg 5-MeO-DMT.
2. The second dose (12 mg) will only be administered if:
   a. A peak experience (total score of ·75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of ·75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-Me-ODMT.
8. Has any current or past clinically significant condition (e.g., severe infection, pulmonary disease, uncontrolled-hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's-judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day DR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in Clinical Global Impression—Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score ·75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

### Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being ·75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 4). The patient also fulfilled standard criteria for MADRS response (at least 50% improvement from baseline) and MADRS remission (MADRS total score equal or less than 10).

TABLE 4
MADRS/BPRS scores table

| | | 2 hr | | | | | |
|---|---|---|---|---|---|---|---|
| | | (MADRS); | | | Improvement | | |
| | Base- | Discharge | | | 2 hr; | | |
| Scale item | line | (BPRS) | Day 1 | Day 7 | Discharge | Day 1 | Day 7 |
| MADRS | | | | | | | |
| Reported sadness | 4 | 0 | 0 | 0 | 4 | 4 | 4 |
| Apparent sadness | 5 | 0 | 0 | 0 | 5 | 5 | 5 |
| Inner Tension | 5 | 0 | 0 | 1 | 5 | 5 | 4 |
| Reduced Sleep | 5 | 5 | 0 | 0 | 0 | 5 | 5 |
| Reduced appetite | 0 | 0 | 0 | 0 | 0 | 0 | 0 |
| Concentration Difficulties | 0 | 0 | 0 | 0 | 0 | 0 | 0 |
| Lassitude | 3 | 0 | 0 | 0 | 3 | 3 | 3 |
| Inability to Feel | 3 | 0 | 0 | 0 | 3 | 3 | 3 |
| Pessimistic Thoughts | 4 | 0 | 0 | 0 | 4 | 4 | 4 |
| Suicidal Thoughts | 0 | 0 | 0 | 0 | 0 | 0 | 0 |
| Total MADRS | 29 | 5 | 0 | 1 | 24 | 29 | 28 |
| BPRS | | | | | | | |
| Somatic Concern | 6 | 1 | 1 | 1 | 5 | 5 | 5 |
| Anxiety | 7 | 1 | 1 | 1 | 6 | 6 | 6 |
| Emotional withdrawal | 5 | 1 | 1 | 1 | 4 | 4 | 4 |
| Conceptual disorganization | 1 | 1 | 1 | 1 | 0 | 0 | 0 |
| Guilt feelings | 6 | 1 | 1 | 1 | 5 | 5 | 5 |
| Tension | 6 | 1 | 1 | 1 | 5 | 5 | 5 |
| Mannerisms and Posturing | 1 | 1 | 1 | 1 | 0 | 0 | 0 |
| Grandiosity | 1 | 1 | 1 | 1 | 0 | 0 | 0 |
| Depressive Mood | 6 | 1 | 1 | 1 | 5 | 5 | 5 |
| Hostility | 1 | 1 | 1 | 1 | 0 | 0 | 0 |
| Suspiciousness | 1 | 1 | 1 | 1 | 0 | 0 | 0 |
| Hallucinatory behaviour | 1 | 1 | 1 | 1 | 0 | 0 | 0 |
| Motor retardation | 1 | 1 | 1 | 1 | 0 | 0 | 0 |
| Uncooperativeness | 1 | 1 | 1 | 1 | 0 | 0 | 0 |
| Unusual Thought Content | 1 | 1 | 1 | 1 | 0 | 0 | 0 |
| Blunted affect | 1 | 1 | 1 | 1 | 0 | 0 | 0 |
| Excitement | 2 | 1 | 1 | 1 | 1 | 1 | 1 |
| Disorientation | 1 | 1 | 1 | 1 | 0 | 0 | 0 |

Significant improvements were noted for several MADRS items in particular. The items are outlined in Table 4. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

Additionally, improvements in maternal functioning were evidenced by improvements in the BIMF score recorded at Day 7, as outlined in Table 5, with the total score improving by 14% from 92 to 105 (out of a possible total of 120).

Several functional domains of maternal function were also assessed, as defined by Barkin et al. The improvements in each functional domain are outlined in more detail in Table 6.

Here, noteworthy improvements in self-care, psychological well-being and management were achieved, with percentage improvements ranging from 18% (management) to (44%),% (self-care). These improvements reinforce the relationship between improvement in depressive items, as assessed by the MADRS, and improvements in maternal functioning.

It is noted that the patient scored comparatively high already before treatment. In some functional domains, the score was at the maximum value, or close to it (see Table 6), so that the scope for improvement by therapy was limited.

TABLE 5
BIMF scores table

| No. | BIM F item | Day 0 | Day 7 | Δ (Day 7) |
|---|---|---|---|---|
| 1 | I am a good mother | 6 | 6 | 0 |
| 2 | I feel rested | 0 | 2 | 2 |
| 3 | I am comfortable with the way I've chosen to feed my | 6 | 6 | 0 |
| | baby (either bottle or breast, or both) | | | |
| 4 | My baby and I understand each other | 5 | 5 | 0 |
| 5 | I am able to relax and enjoy time with my baby | 5 | 6 | 1 |
| 6 | There are people in my life that I can trust to care for | 6 | 6 | 0 |
| | my baby when I need a break | | | |
| 7 | I am comfortable allowing a trusted friend or relative | 6 | 6 | 0 |
| | to care for my baby (can include baby's father or | | | |
| | partner) | | | |
| 8 | I am getting enough adult interaction | 6 | 6 | 0 |
| 9 | I am getting enough encouragement from other people | 5 | 6 | 1 |
| 10 | I trust my own feelings (instincts) when it comes to | 5 | 6 | 1 |
| | taking care of my baby | | | |
| 11 | I take a little time each week to do something for | 5 | 6 | 1 |
| | myself | | | |
| 12 | I am taking good care of my baby's physical needs | 6 | 6 | 0 |
| | (feedings, changing diapers, doctor's appointments) | | | |
| 13 | I am taking good care of my physical needs (eating, | 4 | 5 | 1 |
| | showering, etc) | | | |
| 14 | I make good decisions about my baby's health and | 6 | 6 | 0 |
| | well being | | | |
| 15 | My baby and I are getting into a routine | 5 | 6 | 1 |
| 16 | I worry about how other people judge me (as a | 3 | 5 | 2 |
| | mother) | | | |
| 17 | I am able to take care of my baby and my other | 5 | 5 | 0 |
| | responsibilities | | | |
| 18 | Anxiety or worry often interferes with my mothering | 2 | 5 | 3 |
| | ability | | | |
| 19 | As time goes on, I am getting better at taking care of | 0 | 1 | 1 |
| | my baby | | | |
| 20 | I am satisfied with the job I am doing as a new mother | 6 | 5 | −1 |
| | TOTAL | 92 | 105 | 13 |

TABLE 6
BIMF functional domain scores table

| | | Score | | | | |
|---|---|---|---|---|---|---|
| | Related BIMF | Maximum | Day | Day | Δ (Day | |
| Functional Domain | Items | Possible | 0 | 7 | 7) | % Improvement |
| Self Care | 2, 11, 13 | 18 | 9 | 13 | 4 | 44.44% |
| Infant Care | 12, 14 | 12 | 12 | 12 | 0 | 0.00% |
| Mother-Child Interaction | 4, 5, 15 | 18 | 15 | 17 | 2 | 13.33% |
| Psychological | 1, 2, 3, 5, 7, 10, 11, | 60 | 44 | 53 | 9 | 20.45% |
| Well-being | 16, 18, 20 | | | | | |
| Social Support | 6, 8, 9 | 18 | 17 | 18 | 1 | 5.88% |
| Management | 7, 11, 13, 14, 17, 18 | 36 | 28 | 33 | 5 | 17.86% |
| Adjustment | 17, 19 | 12 | 5 | 6 | 1 | 20.00% |

### Summary and Conclusions

A. An individualised dosing regimen of 6 mg 5-MeO-DMT, followed by 12 mg 5-Me-ODMT administered via inhalation was well tolerated and induced an astonishing and very significant clinical response in a patient formally diagnosed with postpartum depression.
B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRls), serotonin-norepinephrine reuptake inhibitors (SNRls), and others, which generally take 4 to 6 weeks to show their effect.
C. The patient experienced a clinical remission within 2 hours after 5-MeO-DMT administration according to the IDR. This is highly superior to any approved therapy for postpartum depression, and also to all previously tested psychedelic agents.
D. A significant clinical response was sustained over the 7-day follow-up period, although 5-MeO-DMT was only given once and is no longer efficaciously present in the body during this time frame (see pharmacokinetic data in Example 6 above).

This observation supports the superior clinical profile of 5-MeO-DMT and allows for convenient administration intervals.
E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.
F. In addition to anti-depressive effects, endpoints assessing maternal functioning, as assessed using the BIMF, such as self-care, psychological well-being and management, were positively impacted. This supports additional benefits of 5-MeO-DMT to patients suffering from PPD beyond improvement in their core depressive symptoms.

The highlighted aspects show that 5-MeO-DMT has a significantly improved efficacy profile compared to approved pharmacological therapies for postpartum depression and to all previously tested psychedelic agents, when used according to the present invention.

Together with the short duration of the acute psychedelic effects and the favourable safety profile, these data show that the technical problem to provide an improved psychoactive therapy in a patient with a postpartum depression is solved by the present invention.

### Example 9—Clinical Trial of 5-MeO-DMT Administered Via Inhalation to Patients with Bipolar II Disorder

The single-arm, open-label clinical trial will involve 15 adult patients with bipolar II disorder and a current major depressive episode.

Patients who are currently taking anti-depressive medication need to discontinue or taper over time such medication.

The patients will receive a single-day individualized 5-MeO-DMT dosing regimen via inhalation after vaporization.

More in particular, the patients will receive up to three doses of 5-MeO-DMT on the administration day (Day 0): 6 mg, 12 mg, and 18 mg.
1. All patients will receive an initial dose of 6 mg 5-MeO-DMT.
2. The second dose (12 mg) will only be administered if:
   a. A peak experience (PES total score of ·75) has not been achieved following the 6 mg dose, and
   b. The 6 mg dose was safe and well-tolerated.
3. Similarly, a third dose (18 mg) will only be administered if:
   a. A peak experience (PES total score of ·75) has not been achieved following the 12 mg dose, and
   b. The 12 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c)·clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-Me-ODMT.
9. Has any current or past clinically significant condition (e.g., severe infection, severe pulmonary disease, uncontrolled-hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 6 mg, 12 mg and 18 mg of 5-MeO-DMT in patients with bipolar II disorder and a current major episode of depression. Secondary objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 6 mg, 12 mg and 18 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via inhalation evaluated by:
   The proportion of patients in remission (MADRS ·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via inhalation evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score ·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 10—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 2 mg, 5 mg, and 8 mg.
1. All patients will receive an initial dose of 2 mg 5-MeO-DMT.
2. The second dose (5 mg) will only be administered if:
   a. A peak experience (PES total score of ·75) has not been achieved following the 2 mg dose, and
   b. The 2 mg dose was safe and well-tolerated.
3. Similarly, a third dose (8 mg) will only be administered if:
   a. A peak experience (PES total score of ·75) has not been achieved following the 5 mg dose, and
   b. The 5 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c)-clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-Me-ODMT.
9. Has any current or past clinically significant condition (e.g., severe infection, severe pulmonary disease, uncontrolled-hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 2 mg, 5 mg and 8 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 2 mg, 5 mg and 8 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS ·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
   The safety and tolerability of 5-MeO-DMT administered via intravenous injection evaluated by:
Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score ·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 11—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 1 mg, 2 mg, and 3 mg.
1. All patients will receive an initial dose of 1 mg 5-MeO-DMT.
2. The second dose (2 mg) will only be administered if:
   a. A peak experience (PES total score of ·75) has not been achieved following the 1 mg dose, and
   b. The 1 mg dose was safe and well-tolerated.
3. Similarly, a third dose (3 mg) will only be administered if:
   a. A peak experience (PES total score of ·75) has not been achieved following the 2 mg dose, and
   b. The 2 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c)-clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-Me-ODMT.
9. Has any current or past clinically significant condition (e.g., severe infection, severe pulmonary disease, uncontrolled-hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 1 mg, 2 mg and 3 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 1 mg, 2 mg and 3 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS ·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score ·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

1. 5-Methoxy-N,N-dimethyltryptamine (5-MeO-DMT) or a pharmaceutically acceptable salt thereof for use in treating a patient suffering from psychomotor retardation, wherein the 5-MeO-DMT is administered via the intravenous, intramuscular or subcutaneous route.

2. 5-MeO-DMT or a pharmaceutically acceptable salt thereof for use as in claim 1, wherein the treatment reduces or eliminates psychomotor retardation.

3. 5-MeO-DMT or a pharmaceutically acceptable salt thereof for use as in claim 2, wherein the reduction or elimination of psychomotor retardation is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

4. 5-MeO-DMT or a pharmaceutically acceptable salt thereof for use as in claim 2, wherein the reduction or elimination of psychomotor retardation occurs not later than about 2 hours after the last administration of 5-MeO-DMT or a pharmaceutically acceptable salt thereof and wherein the reduction or elimination of psychomotor retardation persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

5. 5-MeO-DMT or a pharmaceutically acceptable salt thereof for use as in claims 1 to 4, wherein the patient is suffering from a mental or nervous system disorder associated with the psychomotor retardation.

6. 5-MeO-DMT or a pharmaceutically acceptable salt thereof for use as in claim 5, wherein the patient suffering from a mental or nervous system disorder suffers from a treatment resistant form of the disorder.

7. 5-MeO-DMT or a pharmaceutically acceptable salt thereof for use as in claims 1 to 4, wherein the patient is suffering from a disorder characterized by depressive episodes associated with the psychomotor retardation.

8. 5-MeO-DMT or a pharmaceutically acceptable salt thereof for use as in claim 7, wherein the patient suffering from a disorder characterized by depressive episodes suffers from a treatment resistant form of the disorder.

9. 5-MeO-DMT or a pharmaceutically acceptable salt thereof for use as in claims 1 to 4, wherein the patient is suffering from major depressive disorder (MDD) associated with the psychomotor retardation.

10. 5-MeO-DMT or a pharmaceutically acceptable salt thereof for use as in claim 9, wherein the patient suffering from MDD suffers from a treatment resistant form of the disorder.

11. 5-MeO-DMT or a pharmaceutically acceptable salt thereof for use as in claims 1 to 4, wherein the patient is suffering from postpartum depression (PPD) associated with the psychomotor retardation.

12. 5-MeO-DMT or a pharmaceutically acceptable salt thereof for use as in claim 11, wherein the patient suffering from PPD suffers from a treatment resistant form of the disorder.

13. 5-MeO-DMT or a pharmaceutically acceptable salt thereof for use as in claims 11 or 12, wherein the patient suffers in addition from compromised maternal functioning.

14. 5-MeO-DMT or a pharmaceutically acceptable salt thereof for use as in claim 13, wherein the patient has a Barkin Index of Maternal Functioning (BIMF) score of 80 or below, such as 65 or below.

15. 5-MeO-DMT or a pharmaceutically acceptable salt thereof for use as in claims 13 or 14, wherein the treatment improves maternal functioning.

16. 5-MeO-DMT or a pharmaceutically acceptable salt thereof for use as in claim 15, wherein the improvement in maternal functioning is reflected by an improvement of the BIMF total score by 10% or more, preferably by 20% or more.

17. 5-MeO-DMT or a pharmaceutically acceptable salt thereof for use as in claims 15 or 16, wherein the improvement in maternal functioning, is reflected by at least an improvement in the BIMF total score on day 7; on day 14; and/or on day 28 after the last administration of 5-MeO-DMT or a pharmaceutically acceptable salt thereof.

18. 5-MeO-DMT or a pharmaceutically acceptable salt thereof for use as in claims 15 or 16, wherein the improvement in maternal functioning, as reflected by at least an improvement in the BIMF total score, occurs not later than about 6 days after the last administration of 5-MeO-DMT or a pharmaceutically acceptable salt thereof and wherein the improvement in maternal functioning, as reflected by at least an improvement in the BIMF total score, persists until at least 14 days after the last administration of 5-MeO-DMT or a pharmaceutically acceptable salt thereof; more preferably until at least 28 days after the last administration of 5-MeO-DMT or a pharmaceutically acceptable salt thereof.

19. 5-MeO-DMT or a pharmaceutically acceptable salt thereof for use as in claims 1 to 4, wherein the patient is suffering from bipolar disorder associated with the psychomotor retardation.

20. 5-MeO-DMT or a pharmaceutically acceptable salt thereof for use as in claim 19, wherein the patient is suffering from bipolar II disorder associated with the psychomotor retardation.

21. 5-MeO-DMT or a pharmaceutically acceptable salt thereof for use as in claim 19, wherein the patient is suffering from bipolar I disorder associated with the psychomotor retardation.

22. 5-MeO-DMT or a pharmaceutically acceptable salt thereof for use as in claims 19 to 21, wherein the patient suffers from a current major depressive episode.

23. 5-MeO-DMT or a pharmaceutically acceptable salt thereof for use as in claims 19 to 22, wherein the patient suffering from bipolar disorder suffers from a treatment resistant form of the disorder.

24. 5-MeO-DMT or a pharmaceutically acceptable salt thereof for use as in claims 1 to 4, wherein the patient is suffering from seasonal affective disorder associated with the psychomotor retardation.

25. 5-MeO-DMT or a pharmaceutically acceptable salt thereof for use as in claim 24, wherein the patient suffering from seasonal affective disorder suffers from a treatment resistant form of the disorder.

26. 5-MeO-DMT or a pharmaceutically acceptable salt thereof for use as in claims 1 to 4, wherein the patient is suffering from persistent depressive disorder associated with the psychomotor retardation.

27. 5-MeO-DMT or a pharmaceutically acceptable salt thereof for use as in claim 26, wherein the patient suffering from persistent depressive disorder suffers from a treatment resistant form of the disorder.

28. 5-MeO-DMT or a pharmaceutically acceptable salt thereof for use as in claims 1 to 4, wherein the patient is suffering from a mental and behavioural disorder due to psychoactive substance use associated with the psychomotor retardation.

29. 5-MeO-DMT or a pharmaceutically acceptable salt thereof for use as in claim 28, wherein the patient is suffering from substance use disorder (SUD) associated with the psychomotor retardation.

30. 5-MeO-DMT or a pharmaceutically acceptable salt thereof for use as in claims 28 or 29, wherein the patient suffers from a treatment resistant form of the disorder.

31. 5-MeO-DMT or a pharmaceutically acceptable salt thereof for use as in claims 1 to 4, wherein the patient is suffering from a psychotic disorder associated with the psychomotor retardation.

32. 5-MeO-DMT or a pharmaceutically acceptable salt thereof for use as in claim 31, wherein the patient suffering from a psychotic disorder suffers from a treatment resistant form of the disorder.

33. 5-MeO-DMT or a pharmaceutically acceptable salt thereof for use as in claims 1 to 4, wherein the patient is suffering from schizophrenia associated with the psychomotor retardation.

34. 5-MeO-DMT or a pharmaceutically acceptable salt thereof for use as in claim 33, wherein the patient suffering from schizophrenia suffers from a treatment resistant form of the disorder.

35. 5-MeO-DMT or a pharmaceutically acceptable salt thereof for use as in claims 1 to 4, wherein the patient is suffering from dementia associated with the psychomotor retardation.

36. 5-MeO-DMT or a pharmaceutically acceptable salt thereof for use as in claims 1 to 4, wherein the patient is suffering from Alzheimer's dementia associated with the psychomotor retardation.

37. 5-MeO-DMT or a pharmaceutically acceptable salt thereof for use as in claims 1 to 4, wherein the patient is suffering from dementia with Lewy Bodies associated with the psychomotor retardation.

38. 5-MeO-DMT or a pharmaceutically acceptable salt thereof for use as in claims 1 to 4, wherein the patient is suffering from vascular dementia associated with the psychomotor retardation.

39. 5-MeO-DMT or a pharmaceutically acceptable salt thereof for use as in claims 1 to 4, wherein the patient is suffering from Parkinson's Disease Dementia associated with the psychomotor retardation.

40. 5-MeO-DMT or a pharmaceutically acceptable salt thereof for use as in claims 1 to 4, wherein the patient is suffering from Parkinson's disease associated with the psychomotor retardation.

41. 5-MeO-DMT or a pharmaceutically acceptable salt thereof for use as in claims 1 to 4, wherein the patient is suffering from Chronic Fatigue Syndrome associated with the psychomotor retardation.

42. 5-MeO-DMT or a pharmaceutically acceptable salt thereof for use as in claim 41, wherein the patient suffering from Chronic Fatigue Syndrome suffers from a treatment resistant form of the disorder.

43. 5-MeO-DMT or a pharmaceutically acceptable salt thereof for use as in claims 5 to 42, wherein the treatment leads to an improvement in the diagnosed disorder in a patient also suffering from associated psychomotor retardation.

44. 5-MeO-DMT or a pharmaceutically acceptable salt thereof for use as in claim 43, wherein the improvement in the diagnosed disorder in a patient also suffering from associated psychomotor retardation, as reflected by a reduction in the Clinical Global Impression-Severity (CGI-S) score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

45. 5-MeO-DMT or a pharmaceutically acceptable salt thereof for use as in claim 43, wherein the improvement in the diagnosed disorder in a patient also suffering from associated psychomotor retardation, as reflected by a reduction in the Clinical Global Impression-Severity (CGI-S) score, occurs not later than about 2 hours after the last administration of 5-MeO-DMT or a pharmaceutically acceptable salt thereof and wherein the improvement in the diagnosed disorder in a patient also suffering from associated psychomotor retardation, as reflected by a reduction in the Clinical Global Impression-Severity (CGI-S) score, persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

46. 5-MeO-DMT or a pharmaceutically acceptable salt thereof for use as in claims 1 to 4, wherein the patient is suffering from a sleep disturbance associated with the psychomotor retardation.

47. 5-MeO-DMT or a pharmaceutically acceptable salt thereof for use as in claim 46, wherein the sleep disturbance is insomnia associated with the psychomotor retardation.

48. 5-MeO-DMT or a pharmaceutically acceptable salt thereof for use as in claims 46 or 47, wherein the patient suffers from an idiopathic sleep disturbance associated with the psychomotor retardation.

49. 5-MeO-DMT or a pharmaceutically acceptable salt thereof for use as in claims 46 to 48, wherein the patient suffers from a treatment resistant form of the disorder.

50. 5-MeO-DMT or a pharmaceutically acceptable salt thereof for use as in claims 46, 47 or 49, wherein the sleep disturbance occurs in patient suffering from associated psychomotor retardation and also from a mental or nervous system disorders, such as disorders characterized by depressive episodes, for example Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Mental and Behavioural Disorders due to Psychoactive Substance Use, for example Substance Use Disorder (SUD); Psychotic Disorders, for example Schizophrenia; Dementia, for example Alzheimer's Dementia (AD); Dementia with Lewy Bodies (DLB); Vascular Dementia and Parkinson's Disease Dementia; Parkinson's Disease (PD); and Chronic Fatigue Syndrome.

51. 5-MeO-DMT or a pharmaceutically acceptable salt thereof for use as in claim 50, wherein the treatment leads to an improvement in psychomotor retardation and sleep disturbance and furthermore to an improvement in the associated mental or the nervous system disorder.

52. 5-MeO-DMT or a pharmaceutically acceptable salt thereof for use as in claims 46 to 51, wherein the treatment leads to an improvement in the sleep disturbance.

53. 5-MeO-DMT or a pharmaceutically acceptable salt thereof for use as in claim 52, wherein the improvement in the sleep disturbance, as reflected by a reduction in the Clinical Global Impression-Severity (CGI-S) score, is observed on day 1, for instance after about 24 hours; on day 7; on day 14; and/or on day 28 after the last administration of 5-MeO-DMT or a pharmaceutically acceptable salt thereof.

54. 5-MeO-DMT or a pharmaceutically acceptable salt thereof for use as in claim 52, wherein the improvement in the sleep disturbance, as reflected by a reduction in the Clinical Global Impression-Severity (CGI-S) score, occurs not later than about 24 hours after the last administration of 5-MeO-DMT or a pharmaceutically acceptable salt thereof and wherein the improvement in the sleep disturbance, as reflected by a reduction in the Clinical Global Impression-Severity (CGI-S) score, persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

55. 5-MeO-DMT or a pharmaceutically acceptable salt thereof for use as in claim 52 wherein the improvement in the sleep disturbance, as reflected by a reduction in the Clinical Global Impression-Severity (CGI-S) score, is observed on day 7 after the last administration of 5-MeO-DMT or a pharmaceutically acceptable salt thereof and preferably persists until at least 14 days, more preferably until at least 28 days.

56. 5-MeO-DMT or a pharmaceutically acceptable salt thereof for use as in claims 1 to 55, wherein the 5-MeO-DMT or salt thereof is administered at a dose or in a dosage regimen that causes the patient to experience a peak psychedelic experience.

57. 5-MeO-DMT or a pharmaceutically acceptable salt thereof for use as in claims 1 to 56, wherein a dosage of about 1 mg to about 10 mg 5-MeO-DMT is administered, or wherein equimolar amounts of the pharmaceutically acceptable salt are administered instead of 5-MeO-DMT.

58. 5-MeO-DMT or a pharmaceutically acceptable salt thereof for use as in claims 1 to 56, wherein a dosage of about 2 mg; or of about 5 mg; or of about 8 mg is administered, or wherein equimolar amounts of the pharmaceutically acceptable salt are administered instead of 5-MeO-DMT.

59. 5-MeO-DMT or a pharmaceutically acceptable salt thereof for use as in claims 1 to 56, wherein a dosage of about 1 mg; or of about 2 mg; or of about 3 mg is administered, or wherein equimolar amounts of the pharmaceutically acceptable salt are administered instead of 5-MeO-DMT

60. 5-MeO-DMT or a pharmaceutically acceptable salt thereof for use as in claims 1 to 57, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

61. 5-MeO-DMT or a pharmaceutically acceptable salt thereof for use as in claims 1 to 57 or 60, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.

62. 5-MeO-DMT or a pharmaceutically acceptable salt thereof for use as in claim 61, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.

63. 5-MeO-DMT or a pharmaceutically acceptable salt thereof for use as in claims 1 to 57 or 60, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.

64. 5-MeO-DMT or a pharmaceutically acceptable salt thereof for use as in claim 63, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.

65. 5-MeO-DMT or a pharmaceutically acceptable salt thereof for use as in claims 60 to 64, wherein the interval between two administrations is not less than 1 hour and not more than 24 hours, such as about 1 to 4 hours, preferably 1 to 2 hours.

66. 5-MeO-DMT or a pharmaceutically acceptable salt thereof for use as in claims 56 to 65, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

67. 5-MeO-DMT or a pharmaceutically acceptable salt thereof for use as in claim 66, wherein the occurrence of a peak psychedelic experience is identified through achievement of a Peak Experience Scale (PES) Total Score of at least 75.

68. 5-MeO-DMT or a pharmaceutically acceptable salt thereof for use as in claims 1 to 67, wherein the 5-MeO-DMT or a pharmaceutically acceptable salt thereof is administered via intravenous injection.
